(19) United States
(12) Patent Application Publication
(10) Pub. No.: US 2011/0237629 A1
(43) Pub. Date: Sep. 29, 2011

(54) AMINO ACID ESTER PRODRUGS AND THE USE THEREOF

(75) Inventors: Daniel Meibom, Leverkusen (DE); Hans-Georg Lerchen, Leverkusen (DE); Alexandros Vakalopoulos, Hilden (DE); Barbara Albrech-Küpper, Wulfrath (DE); Peter Nell, Woodside, CA (US); Jörg Keldenich, Wuppertal (DE); Katja Zimmermann, Dusseldorf (DE); Ursula Krenz, Leichlingen (DE)

(73) Assignee: Bayer Schering Pharma Aktiengesellschaft, Berlin (DE)

(21) Appl. No.: 13/132,989

(22) PCT Filed: Dec. 3, 2009
(86) PCT No.: PCT/EP09/08618

§ 371 Date: Jun. 6, 2011

(30) Foreign Application Priority Data
Dec. 16, 2008 (DE) 10 2008 062 566.3

(51) Int. Cl. A61K 31/4439; C07D 413/12; A61P 9/00; A61P 9/04; A61P 9/12; A61P 9/10

(52) U.S. Cl. 514/340; 546/271.4

(57) ABSTRACT

The present application relates to amino acid ester prodrug derivatives of 2-amino-6-({[2-(4-chlorophenyl)-1,3-oxazol-4-yl]methyl}sulfanyl)-4-(4-{[2,3-dihydroxypropyl]oxy}phenyl)pyridine-3,5-dicarbonitriles, processes for their preparation, their use for the treatment and/or prophylaxis of diseases, and their use for the manufacture of medicaments for the treatment and/or prophylaxis of diseases, especially of cardiovascular disorders.

The present application relates to amino acid ester prodrug derivatives of 2-amino-6-({[2-(4-chlorophenyl)-1,3-oxazol-4-yl]methyl}sulfanyl)-4-(4-{[2,3-dihydroxypropyl]oxy}phenyl)pyridine-3,5-dicarbonitriles, processes for their preparation, their use for the treatment and/or prophylaxis of diseases, and their use for the manufacture of medicaments for the treatment and/or prophylaxis of diseases, especially of cardiovascular disorders.

Prodrugs are derivatives of an active ingredient which undergo in vivo an enzymatic and/or chemical biotransformation in one or more stages before the actual active ingredient is liberated. A prodrug residue is ordinarily used in order to improve the profile of properties of the underlying active ingredient [P. Ettmayer et al., J. Med. Chem. 47, 2393-2404 (2004)]. In order to achieve an optimal profile of effects, it is necessary, in this connection, for the design of the prodrug residue, as well as the desired mechanism of liberation, to conform very accurately with the individual active ingredient, the indication, the site of action, and the administration route. A large number of medicaments are administered as prodrugs which exhibit an improved bioavailability by comparison with the underlying active ingredient, for example achieved by improving the physicochemical profile, specifically the solubility, the active or passive absorption properties or the tissue-specific distribution. An example which may be mentioned from the wide-ranging literature on prodrugs is: H. Bundgaard (Ed.), Design of Prodrugs: Bioreversible derivatives for various functional groups and chemical entities, Elsevier Science Publishers B.V., 1985.

Adenosine, a purine nucleoside, is present in all cells and is released under a large number of physiological and pathophysiological stimuli. Adenosine is produced inside cells on degradation of adenosine 5′-monophosphate (AMP) and S-adenosylhomocysteine as intermediate, but can be released from the cell and then exerts, by binding to specific receptors, effects as hormone-light substance or neurotransmitter. To date, the receptor subtypes A1, A2a, A2b and A3 are known [cf. K. A. Jacobson and Z.-G. Gao, Nat. Rev. Drug Discover. 5, 247-264 (2006)]. The activation of A1 receptors by specific A1 agonists leads in humans to a frequency-dependent lowering of the heart rate, without having an effect on the blood pressure. Selective A1 agonists could therefore be suitable, among other things, for the treatment of angina pectoris and atrial fibrillation.

The activation of A2b receptors by adenosine or specific A2b agonists leads to a lowering of blood pressure via the expansion of vessels. The lowering of blood pressure is accompanied by a reflectory increase in heart rate. The increase in heart rate can be reduced by the activation of A1 receptors by specific A1 agonists.

The combined effect of selective A1/A2b agonists on the vascular system and the heart rate therefore results in a systemic lowering of blood pressure without a relevant increase in heart rate. With a pharmacological profile of this kind, dual A1/A2b agonists could be used to treat, for example, hypertension in humans.

2-Amino-6-({[2-(4-chlorophenyl)-1,3-oxazol-4-yl]methyl}sulfanyl)-4-(4-{[2,3-di-hydroxypropyl]oxy}phenyl)pyridine-3,5-dicarbonitriles of the formula (A)

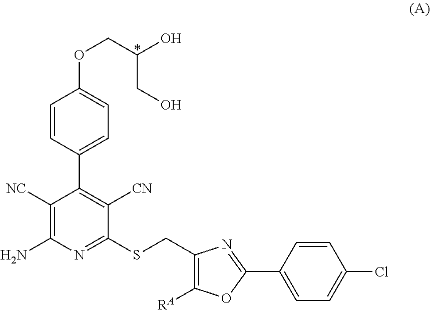

in which

R^{A} is hydrogen or methyl,

are potent and selective adenosine A1 receptor agonists with a certain dual, A2b-agonist component to its action (see PCT application WO 2009/015776-A1). The compounds are presently undergoing in-depth investigation as a possible new active pharmaceutical ingredients for the prevention and therapy of, in particular, cardiovascular disorders. Of particular significance in this context is the compound of the formula (A), in which R^{A} is hydrogen and the C* carbon atom of the propane-1,2-diol group possesses an R-configuration.

However, the compounds of the formula (A) have only a limited solubility in water, physiological media and organic solvents, and an only low bioavailability after oral administration of a suspension of crystalline material. On the one hand, this allows intravenous administration of the active ingredient only in very low dosages; infusion solutions based on physiological saline solutions can be produced only with difficulty with conventional solubilizers. On the other hand formulation in tablet form is difficult. It was therefore an object of the present invention to identify derivatives or prodrugs of compounds of the formula (A) which have an improved solubility in the media mentioned and/or an improved bioavailability after oral administration and, at the same time, make it possible to have controlled liberation of the active ingredient in question in the patient's body after administration. In addition, further areas of therapeutic use of this class of active ingredients could be opened up by an improved possibility of intravenous administration.

A review of prodrug derivatives based on carboxylic esters and possible properties of such compounds is given for example in K. Beaumont et al., Curr. Drug Metab. 4, 461-485 (2003).

The present invention relates to compounds of the general formula (I)

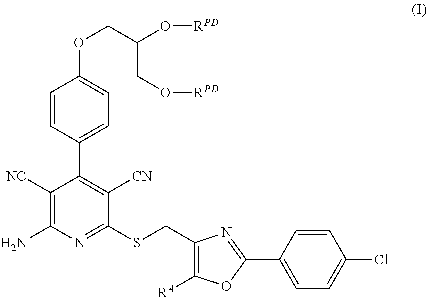

in which

R^{A} is hydrogen or methyl,

and

R^{PD} is a group of the formula

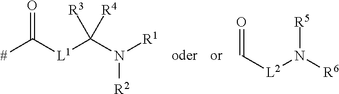

in which

# means the point of linkage to the respective O atom,

L¹ is a bond or —CH₂—,

L² is straight-chain (C₃-C₆)-alkanediyl, which may be substituted up to two times, identically or differently, by (C₁-C₄)-alkyl, hydroxyl and/or (C₁-C₄)-alkoxy,

R¹ and R² are identical or different and independently of one another are hydrogen or (C₁-C₄)-alkyl which may be substituted by hydroxyl, (C₁-C₄)-alkoxy, amino, mono-(C₁-C₄)-alkylamino or di-(C₁-C₄)-alkylamino

or

R¹ and R² are linked to one another and, together with the nitrogen atom to which they are attached, form a 5- or 6-membered saturated heterocycle which may comprise a further ring heteroatom from the series consisting of N and O, and may be substituted one or two times, identically or differently, by (C₁-C₄)-alkyl, amino, hydroxyl and/or (C₁-C₄)-alkoxy,

R³ is hydrogen or the side group of a natural α-amino acid or its homologs or isomers,

or

R³ is linked to R¹ and both, together with the atoms to which they are attached, form a 5- or 6-membered saturated heterocycle which may be substituted once or twice by identical or different (C₁-C₄)-alkyl, amino, hydroxyl and/or (C₁-C₄)-alkoxy substituents, R⁴ is hydrogen or methyl

or

R³ and R⁴ are linked to one another and, together with the carbon atom to which they are attached, form a 3- to 6-membered saturated carbocycle,

and

R⁵ and R⁶ are identical or different and independently of one another are hydrogen or (C1-C4)-alkyl which may be substituted by hydroxyl, (C1-C4)-alkoxy, amino, mono-(C1-C4)-alkylamino or di-(C1-C4)-alkylamino,

and the salts, solvates and solvates of the salts thereof.

Compounds according to the invention are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

The compounds according to the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

Where the compounds according to the invention can occur in tautomeric forms, the present invention encompasses all tautomeric forms.

Salts preferred for the purposes of the present invention are physiologically acceptable salts of the compounds according to the invention. However, salts which are themselves unsuitable for pharmaceutical applications but can be used for example for isolating or purifying the compounds according to the invention are also encompassed. Besides monosalts, the present invention also includes where appropriate possible polysalts such as di- or trisalts.

Physiologically acceptable salts of the compounds according to the invention include acid addition salts of mineral acids, carboxylic acids and sulphonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of usual bases such as, by way of example and preferably, alkali metal salts (e.g. sodium and potassium salts), alkaline earth metal salts (e.g. calcium and magnesium salts) and ammonium salts, derived from ammonia or organic amines having 1 to 16 C atoms, such as, by way of example and preferably, ethylamine, diethylamine, triethylamine, ethyldiisopropylamine, monoethanolamine, diethanolamine, triethanolamine, choline, dicyclohexylamine, dimethylaminoethanol, procain, dibenzylamine, morpholine, N-methylmorpholine, arginine, lysine, ethylenediamine, piperidine and N-methylpiperidine.

Solvates refer for the purposes of the invention to those forms of the compounds according to the invention which form a complex in the solid or liquid state through coordination with solvent molecules. Hydrates are a specific form of solvates in which the coordination takes place with water. Solvates preferred in the context of the present invention are hydrates. In the context of the present invention, the substituents have the following meaning unless otherwise specified:

(C1-C4)-Alkyl is in the context of the invention a straight-chain or branched alkyl radical having 1 to 4 carbon atoms. Examples which may be preferably mentioned are: methyl, ethyl, n-propyl, iopropyl, n-butyl, isobutyl, sec-butyl, tert-butyl.

(C₃-C₆)-Alkanediyl and (C₃-C₅-alkanediyl) are in the context of the invention a straight-chain, α,ω-divalent alkyl radical having 3 to 6 or 3 to 5 carbon atoms. Examples which may be preferably mentioned are: propane-1,3-diyl (1,3-propylene), butane-1,4-diyl (1,4-butylene), pentane-1,5-diyl (1,5-pentylene), hexane-1,6-diyl (1,6-hexylene).

(C₁-C₄)-Alkoxy is in the context of the invention a straight-chain or branched alkoxy radical having 1 to 4 carbon atoms. Examples which may be preferably mentioned are: methoxy, ethoxy, n-propoxy, isopropoxy, n-butoxy, tert-butoxy.

Mono-(C₁-C₄)-alkylamino is in the context of the invention an amino group having a straight-chain or branched alkyl substituent which has 1 to 4 carbon atoms. Examples which may be preferably mentioned are: methylamino, ethylamino, n-propylamino, isopropylamino, n-butylamino, tert-butylamino.

Di-(C₁-C₄)-alkylamino is in the context of the invention an amino group having two, identical or different, straight-chain or branched alkyl substituents which each have 1 to 4 carbon atoms. Examples which may be preferably mentioned are: N,N-dimethylamino, N,N-diethylamino, N-ethyl-N-methylamino, N-methyl-N-n-propylamino, N-isopropyl-N-n-propylamino, N,N-diisopropylamino, N-n-butyl-N-methylamino, N-tert-butyl-N-methylamino.

A 3- to 6-membered carbocycle is in the context of the invention a monocyclic, saturated cycloalkyl group having 3 to 6 ring carbon atoms. Examples which may be preferably mentioned are: cyclopropyl, cyclobutyl, cyclopentyl, cyclohexyl.

A 5- or 6-membered heterocycle is in the context of the invention a monocyclic, saturated heterocycloalkyl group having a total of 5 or 6 ring atoms which contains one ring nitrogen atom and optionally a second ring heteroatom from the series consisting of N and O. Examples which may be preferably mentioned are: pyrrolidinyl, piperidinyl, piperazinyl, morpholinyl.

The side group of an α-amino acid in the meaning of R³ encompasses both the side groups of naturally occurring α-amino acids and the side groups of homologs and isomers of these α-amino acids. The α-amino acid may in this connection have both the L and the D configuration or else be a mixture of the L form and D form. Examples of side groups which may be mentioned are: methyl (alanine), propan-2-yl (valine), propan-1-yl (norvaline), 2-methylpropan-1-yl(leucine), 1-methylpropan-1-yl (isoleucine), butan-1-yl (norleucine), tert-butyl(2-tert-butylglycine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-yl (lysine), 4-amino-3-hydroxybutan-1-yl(hydroxylysine), 3-aminopropan-1-yl(ornithine), 3-guanidinopropan-1-yl (arginine), 3-ureidopropan-1-yl (citrulline). Preferred α-amino acid side groups in the meaning of R³ are methyl (alanine), propan-2-yl (valine), 2-methylpropan-1-yl (leucine), benzyl (phenylalanine), imidazol-4-ylmethyl (histidine), hydroxymethyl (serine), 1-hydroxyethyl (threonine), 4-aminobutan-1-yl(lysine), 3-aminopropan-1-yl(ornithine), 2-aminoethyl (2,4-diaminobutyric acid), aminomethyl(2,3-diaminopropionic acid), 3-guanidinopropan-1-yl (arginine). The L configuration is preferred in each case. In the context of the present invention it is the case that, for all radicals which occur two or more times, their meaning is independent of one another. If radicals in the compounds according to the invention are substituted, the radicals, unless specified otherwise, may be substituted one or more times. In this context, substitution by one or by two identical or different substituents is preferred; particularly preferred is substitution by one substituent.

Preference is given, in the context of the present invention, to compounds of the formula (I) in which

R^{A} is hydrogen or methyl

and

R^{PD} is a group of the formula

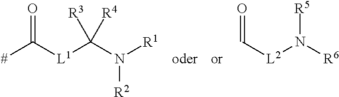

in which

# means the point of linkage to the respective O atom,

L¹ is a bond or —CH₂—,

L² is straight-chain (C₃-C₆)-alkanediyl,

R¹ and R² are independently of one another hydrogen or methyl,

R³ is hydrogen, methyl, propan-2-yl, 2-methylpropan-1-yl, benzyl, imidazol-4-ylmethyl, hydroxymethyl, 1-hydroxyethyl, 4-aminobutan-1-yl, 3-aminopropan-1-yl, 2-aminoethyl, aminomethyl or 3-guanidinopropan-1-yl

or

R³ is linked to R¹ and both, together with the atoms to which they are attached, form a pyrrolidine ring,

R⁴ is hydrogen

and

R⁵ and R⁶ are independently of one another hydrogen or methyl,

and the salts, solvates and solvates of the salts thereof.

Particular preference is given in the context of the present invention to compounds of the formula (I) in which

R^{A} is hydrogen

and

R^{PD} is a group of the formula

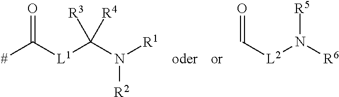

in which

# is the point of linkage to the respective O atom,

L¹ is a bond,

L² is straight-chain (C₃-C₅)-alkanediyl,

R¹ and R² are in each case hydrogen,

R³ is hydrogen, methyl, propan-2-yl, 2-methylpropan-1-yl, hydroxymethyl, 1-hydroxyethyl, 4-aminobutan-1-yl, 3-aminopropan-1-yl, 2-aminoethyl, aminomethyl or 3-guanidinopropan-1-yl,

R⁴ is hydrogen

and

R⁵ and R⁶ are in each case hydrogen,

and the salts, solvates and solvates of the salts thereof.

The two prodrug groups R^{PD} in the compounds of the formula (I) may be identical or different within the scope of the meanings indicated above. Preferred compounds of the formula (I) are those with prodrug groups R^{PD} that are identical in each case.

Of particular importance are the compounds of the formulae (I-A) and (I-B)

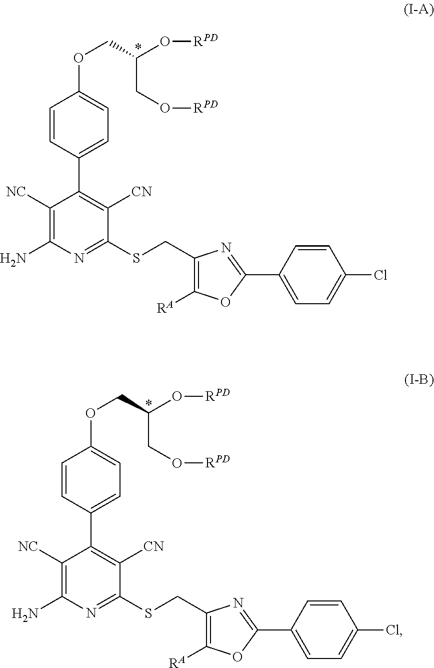

in which R^{A} and R^{PD} have the meaning indicated above, with an S- or R-configuration on the C* carbon atom of the propane-1,2,3-triyl group, and also the salts, solvates and solvates of the salts thereof.

Preferred in the context of the present invention are the compounds of the formula (I-A) with an S-configuration on the C* carbon atom of the propane-1,2,3-triyl group, and also the salts, solvates and solvates of the salts thereof.

Particularly preferred in the context of the present invention are compounds of the formula (I-A) in which R^{A} is hydrogen and the two prodrug groups R^{PD} are each identical, and also the salts, solvates and solvates of the salts thereof.

Further provided by the invention is a process for preparing the compounds of the formula (I) according to the invention in which the two prodrug groups R^{PD} are each identical, characterized in that

[A] the compound of the formula (A)

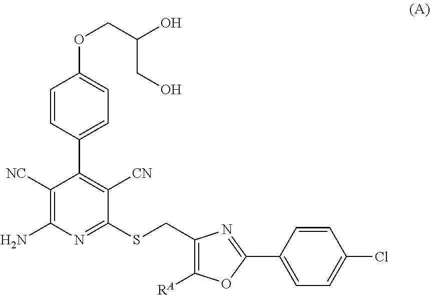

in which

R^{A} is hydrogen or methyl

is coupled in an inert solvent with two or more equivalents of a compound of the formula (II)

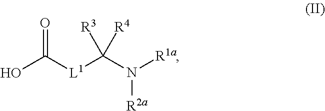

in which L¹, R³ and R⁴ have the meanings indicated above

and

R^{1a} and R^{2a} are identical or different and have the meanings indicated above for R¹ and R², respectively, or are a temporary amino-protective group, with activation of the carboxyl group in (II), to give a compound of the formula (III)

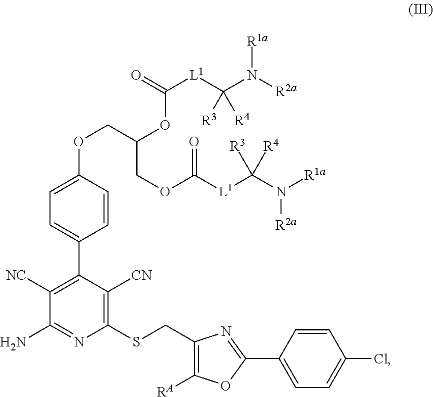

in which L¹, R^{A}, R^{1a}, R^{2a}, R³ and R⁴ have the meanings indicated above, and then any protective groups present are removed, to give a compound of the formula (I-C)

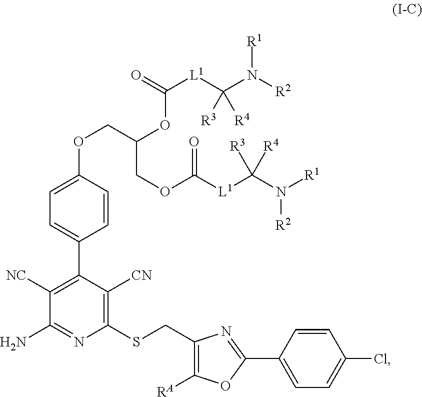

in which L¹, R^{A}, R¹, R², R³ and R⁴ have the meanings indicated above,

or

[B] the compound of the formula (A) is coupled in an inert solvent with two or more equivalents of a compound of the formula (IV)

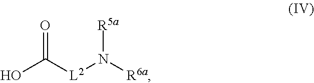

in which L² has the meaning indicated above

and

R^{5a} and R^{6a} are identical or different and have the meanings indicated above for R⁵ and R⁶, respectively, or are a temporary amino-protective group, with activation of the carboxyl group in (IV), to give a compound of the formula (V)

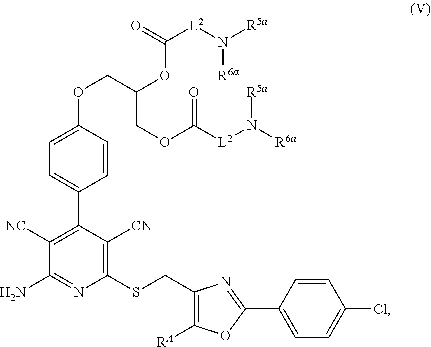

and then any protective groups present are removed, to give a compound of the formula (I-D)

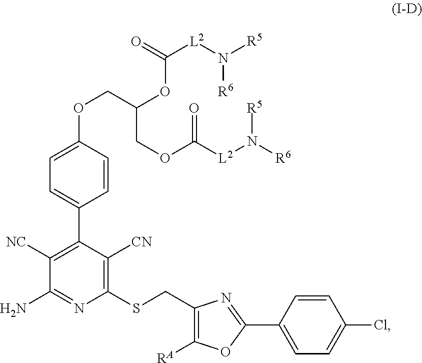

in which L², R⁴, R^{5a} and R^{6a} have the meanings indicated above, and the resulting compounds of the formula (I-C) or (I-D) are converted where appropriate with the appropriate (i) solvents and/or (ii) acids or bases into the solvates, salts and/or solvates of the salts thereof.

The compounds of the formulae (I-C) and (I-D), when prepared in accordance with the processes described above, may also be formed directly in the form of salts. These salts can be converted optionally by treatment with a base and/or acid in an inert solvent, via chromatographic methods or by means of ion exchange resins, into the respective free bases and/or acids. Other salts of the compounds according to the invention may also be prepared, optionally, by replacement of counterions by means of ion exchange chromatography, with Amberlite® resins, for example.

Functional groups present optionally in the compounds of the formulae (II) and (IV), and in the radicals R¹, R², R³, R⁵, R⁶ and/or L²—such as, more particularly, amino, guanidino, hydroxyl, mercapto and carboxyl functional groups—may also be present in a temporarily protected form, if useful or necessary, in the course of the reaction sequences described above. The introduction and removal of such protective groups takes place here in accordance with customary methods known from peptide chemistry [see, for example, T. W. Greene and P. G. M. Wuts, Protective Groups in Organic Synthesis, Wiley, New York, 1999; M. Bodanszky and A. Bodanszky, The Practice of Peptide Synthesis, Springer-Verlag, Berlin, 1984].

An amino-protective and guanidino-protective group used with preference is tert-butoxycarbonyl (Boc) or benzyloxycarbonyl (Z). A protective group used for a hydroxyl or carboxyl function is preferably tert-butyl or benzyl. These protective groups are eliminated by customary methods, preferably by reaction with a strong acid such as hydrogen chloride, hydrogen bromide or trifluoroacetic acid in an inert solvent such as dioxane, dichloromethane or acetic acid; elimination may optionally also take place without an additional inert solvent. In the case of benzyl and benzyloxycarbonyl as protective groups, they may also be removed by hydrogenolysis in the presence of a palladium catalyst. The elimination of the stated protective groups may be undertaken optionally simultaneously in a one-pot reaction, or in separate reaction steps.

Examples of inert solvents for the coupling reaction (ester formation) in the process step (A)+(II)→(III) or (A)+(IV)→(V) are ethers such as diethyl ether, tert-butyl methyl ether, dioxane, tetrahydrofuran, glycol dimethyl ether or diethylene glycol dimethyl ether, hydrocarbons such as benzene, toluene, xylene, hexane, cyclohexane or petroleum fractions, halohydrocarbons such as dichloromethane, trichloromethane, tetrachloromethane, 1,2-dichloroethane, trichloroethylene or chlorobenzene, or other solvents such as acetone, ethyl acetate, pyridine, dimethyl sulfoxide, dimethylformamide, N,N′-dimethylpropyleneurea (DMPU), N-methylpyrrolidone (NMP) or acetonitrile. It is likewise possible to use mixtures of the solvents mentioned.

Dichloromethane, dimethylformamide or mixtures of these two solvents are preferred. Examples suitable for activating the carboxyl group in compound (II) or (IV) in these coupling reactions are carbodiimides such as N,N′-diethyl-, N,N′-dipropyl-, N,N′-diisopropyl-, N,N′-dicyclohexylcarbodiimide (DCC) or N-(3-dimethylaminoisopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium 3-sulfate or 2-tert-butyl-5-methylisoxazolium perchlorate, acylamino compounds such as 2-ethoxy-1-ethoxycarbonyl-1,2-dihydroquinoline, or isobutyl chloroformate, propanephosphonic anhydride, diethyl cyanophosphonate, bis-(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), where appropriate in combination with further auxiliaries such as 1-hydroxybenzotriazole (HOBt) or N-hydroxysuccinimide (HOSu), and as bases are alkali metal carbonates, e.g. sodium or potassium carbonate, or organic amine bases such as triethylamine, N-methylmorpholine, N-methylpiperidine, N,N-diisopropylethylamine or 4-N,N-dimethylaminopyridine. N-(3-Dimethylaminoisopropyl)-N′-ethylcarbodiimide hydrochloride (EDC) in combination with 4-N,N-dimethylaminopyridine is preferably used.

The reactions (A)+(II)→(III) and (A)+(IV)→(V) are generally carried out in a temperature range from 0° C. to +60° C., preferably at +10° C. to +30° C. The reactions can take place under normal, under elevated or under reduced pressure (e.g. from 0.5 to 5 bar). They are generally carried out under atmospheric pressure. The compounds of the formulae (II) and (IV) are commercially available, known from the literature, or can be prepared by methods customary in the literature. Thus, for example, compounds of the formula (II) in which L¹ is —CH₂— can be obtained by known methods for the chain extension of carboxylic acids, such as, for example, the Arndt-Eistert reaction [Eistert et al., Ber. Dtsch. Chem. Ges. 60, 1364-1370 (1927); Ye et al., Chem. Rev. 94, 1091-1160 (1994); Cesar et al., Tetrahedron Lett. 42, 7099-7102 (2001)], from compounds of the formula (II) in which L¹ is a bond.

Compounds of the formula (I) according to the invention in which the two prodrug groups R^{PD} are not identical can be prepared, in analogy to the process described above, by coupling the compound of the formula (A) in sequence with in each case one equivalent of correspondingly different compounds of the formula (II) and/or (IV) and then separating—where appropriate before or after the elimination of temporary protective groups—product mixtures that are produced in these coupling reactions into the individual components. For such separation it is preferred to use chromatographic methods, such as chromatography on silica gel or alumina or else HPLC chromatography on reversed phases, or recrystallization from aqueous or nonaqueous solvent mixtures.

The 2-amino-6-({[2-(4-chlorophenyl)-1,3-oxazol-4-yl]methyl}sulfanyl)-4-(4-{[2,3-dihydroxy-propyl]oxy}phenyl)pyridine-3,5-dicarbonitriles of the formula (A) are prepared by first condensing the benzaldehyde of the formula (VI)

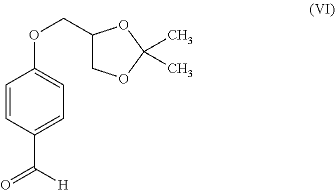

with two equivalents of 2-cyanothioacetamide in the presence of a base such as N-methylmorpholine to give the pyridine derivative (VII)

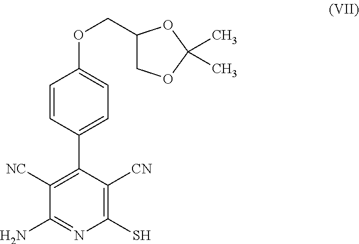

then alkylating this compound in the presence of a base such as sodium hydrogencarbonate with 4-(chloromethyl)-2-(4-chlorophenyl)-1,3-oxazole of the formula (VIII)

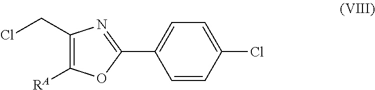

in which R^{A} has the meaning indicated above,

to give a compound of the formula (IX)

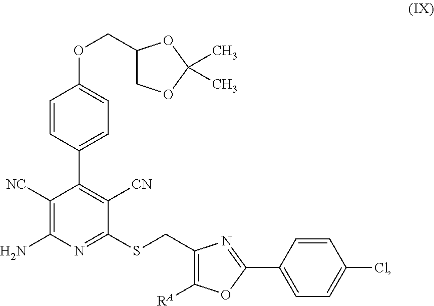

and finally eliminating the acetonide protective group by means of an aqueous acid, such as hydrochloric acid or acetic acid [see also reaction scheme 2 below, and also the description of intermediates 1A-9A and 25A-28A in the Experimental section]. The compound of the formula (VI) in turn is obtainable by reaction of 4-hydroxybenzaldehyde with the 3-chloro-1,2-propanediol acetonide of the formula (X)

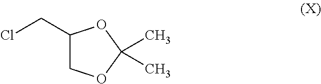

in the presence of a base such as potassium carbonate. If, in this reaction, the enantiomerically pure 3-chloro-1,2-propanediol acetonides in R- or S-configuration are used, then, in accordance with the above-described reaction sequence, it is possible to obtain the corresponding enantiomers of the active ingredient compounds (A) and also, derived from them, the corresponding prodrug compounds of the formulae (I-A) and (I-B).

The 2-phenyl-1,3-oxazole derivatives of the formula (VIII) can be prepared via condensation reactions that are known from the literature [see reaction scheme 3 below].

The preparation of the compounds (I) and of active ingredient compounds (A) according to the invention can be illustrated by way of example by the following synthesis schemes:

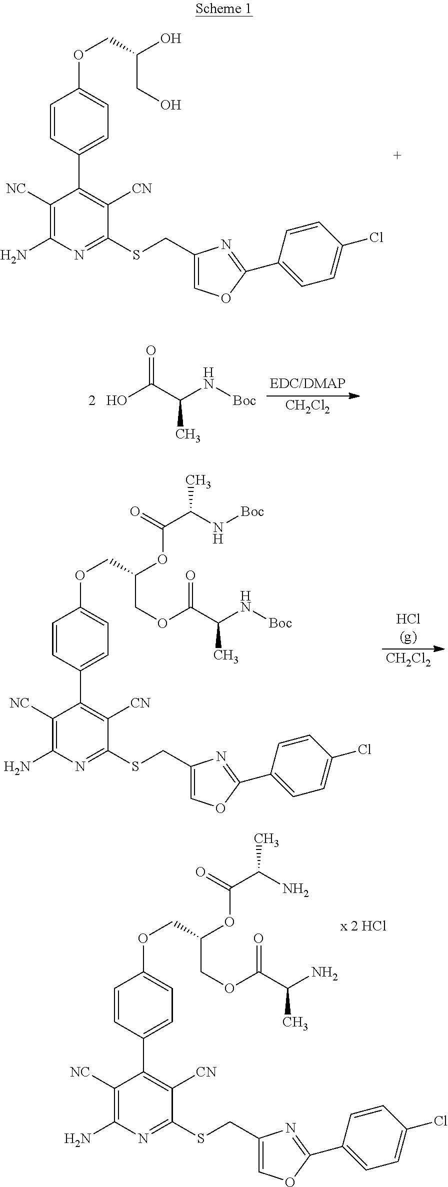

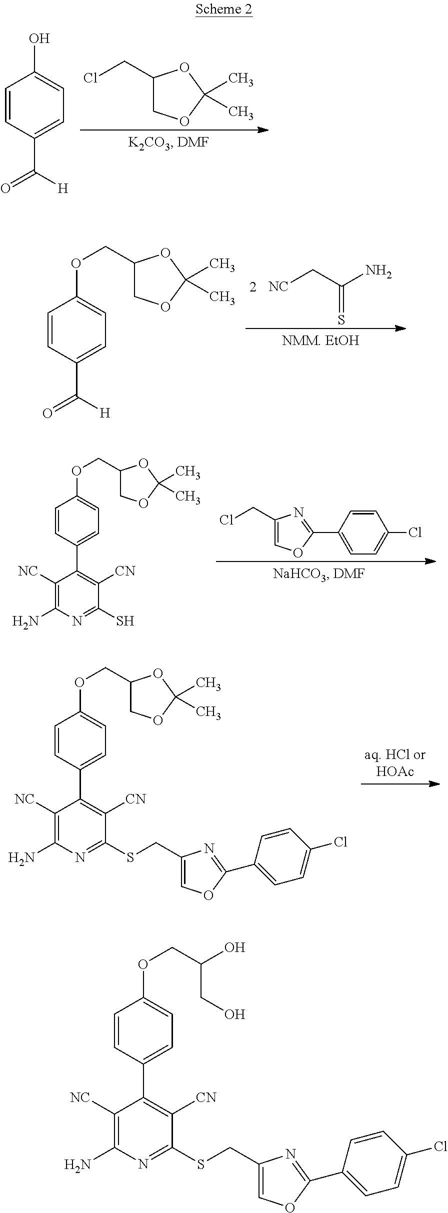

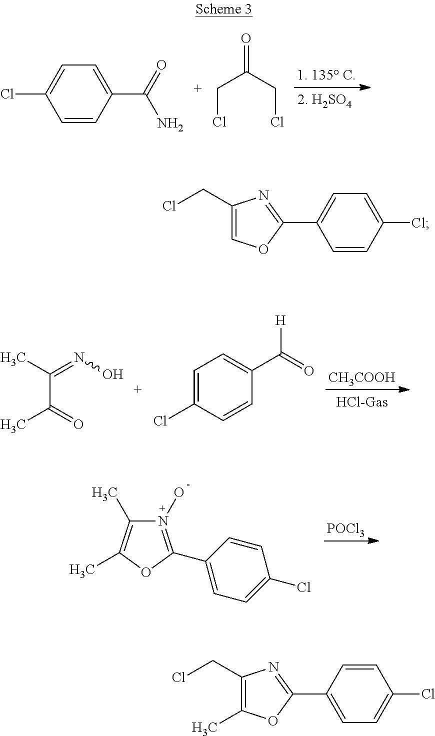

[cf., for example, Y. Goto et al., Chem. Pharm. Bull. 1971, 19, 2050-2057].

The compounds according to the invention and their salts represent useful prodrugs of the active substances of the formula (A). On the one hand, they show good stability at various pH values and, on the other hand, they show efficient conversion into the active ingredient compound (A) at a physiological pH and in particular in vivo. The compounds according to the invention moreover have improved solubilities in aqueous or other physiologically tolerated media, making them suitable for therapeutic use, in particular on intravenous administration. In addition, the bioavailability from suspension after oral administration is improved by comparison with the parent substance (A). The compounds of the formula (I) are suitable alone or in combination with one or more other active ingredients for the prophylaxis and/or treatment of various disorders, for example and in particular disorders of the cardiovascular system (cardiovascular disorders), for cardio protection following lesions of the heart, and of metabolic disorders.

Disorders of the cardiovascular system, or cardiovascular disorders, mean in the context of the present invention for example the following disorders: hypertension (high blood pressure), peripheral and cardiac vascular disorders, coronary heart disease, coronary restenosis such as, for example, restenosis following balloon dilatation of peripheral blood vessels, myocardial infarction, acute coronary syndrome, acute coronary syndrome with ST elevation, acute coronary syndrome without ST elevation, stable and unstable angina pectoris, myocardial insufficiency, Prinzmetal angina, persistent ischemic dysfunction (“hibernating myocardium”), temporary postischemic dysfunction (“stunned myocardium”), heart failure, tachycardia, atrial tachycardia, arrhythmias, atrial and ventricular fibrillation, persistent atrial fibrillation, permanent atrial fibrillation, atrial fibrillation with normal left ventricular function, atrial fibrillation with impaired left ventricular function, Wolff-Parkinson-White syndrome, disturbances of peripheral blood flow, elevated levels of fibrinogen and of low density LDL, and elevated concentrations of plasminogen activator inhibitor 1 (PAI-1), especially hypertension, coronary heart disease, acute coronary syndrome, angina pectoris, heart failure, myocardial infarction and atrial fibrillation.

In the context of the present invention, the term heart failure includes both acute and chronic manifestations of heart failure, as well as more specific or related types of disease, such as acute decompensated heart failure, right heart failure, left heart failure, global failure, ischemic cardiomyopathy, dilated cardiomyopathy, congenital heart defects, heart valve defects, heart failure associated with heart valve defects, mitral stenosis, mitral insufficiency, aortic stenosis, aortic insufficiency, tricuspid stenosis, tricuspid insufficiency, pulmonary stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, and diastolic and systolic heart failure.

The compounds according to the invention are further also suitable in particular for reducing the area of myocardium affected by an infarction, and for the prophylaxis of secondary infarctions.

The compounds according to the invention are furthermore suitable in particular for the prophylaxis and/or treatment of thromboembolic disorders, reperfusion damage following ischemia, micro- and macromuscular lesions (vasculitis), arterial and venous thromboses, edemas, ischemias such as myocardial infarction, stroke and transient ischemic attacks, for cardio protection in connection with coronary artery bypass operations (CABG), primary percutaneous transluminal coronary angioplasties (PTCAs), PTCAs after thrombolysis, rescue PTCA, heart transplants and open-heart operations, and for organ protection in connection with transplants, bypass operations, catheter investigations and other surgical procedures.

Further indication areas for which the compounds according to the invention can be used are for example the prophylaxis and/or treatment of disorders of the urogenital region, such as, for example, acute renal failure, unstable bladder, urogenital incontinence, erectile dysfunction and female sexual dysfunction, but also the prophylaxis and/or treatment of inflammatory disorders such as, for example, inflammatory dermatoses and arthritis, especially rheumatoid arthritis, of disorders of the central nervous system and neurodegenerative impairments (post-stroke conditions, Alzheimer's disease, Parkinson's disease, dementia, Huntington's chorea, epilepsy, depression, multiple sclerosis), of painful conditions and migraine, hepatic fibrosis and cirrhosis of the liver, of cancers and of nausea and vomiting in connection with cancer therapies, and for wound healing.

A further indication area is for example the prophylaxis and/or treatment of respiratory disorders such as, for example, asthma, chronic obstructive respiratory disorders (COPD, chronic bronchitis), pulmonary emphysema, bronchiectasies, cystic fibrosis (mucoviscidosis) and pulmonary hypertension, especially pulmonary aterial hypertension.

Finally, the compounds according to the invention are also suitable for the prophylaxis and/or treatment of metabolic disorders such as, for example, diabetes, especially diabetes mellitus, gestational diabetes, insulin-dependent diabetes and non-insulin-dependent diabetes, diabetic sequelae such as, for example, retinopathy, nephropathy and neuropathy, metabolic disorders such as, for example, metabolic syndrome, hyperglycemia, hyperinsulinemia, insulin resistance, glucose intolerance and obesity (adiposity), and arteriosclerosis and dyslipidemias (hypercholesterolemia, hypertriglyceridemia, elevated concentrations of post-prandial plasma triglycerides, hypoalphalipoproteinemia, combined hyperlipidemias), especially, of diabetes, metabolic syndrome and dyslipidemias.

The present invention further relates to the use of the compounds according to the invention for the treatment and/or prophylaxis of disorders, especially of the aforementioned disorders.

The present invention further relates to the use of the compounds according to the invention for the manufacture of a medicament for the treatment and/or prophylaxis of disorders, especially of the aforementioned disorders.

The present invention further relates to the use of the compounds according to the invention in a method for the treatment and/or prophylaxis of disorders, especially of the aforementioned disorders.

The present invention further relates to a method for the treatment and/or prophylaxis of disorders, especially of the aforementioned disorders, by using an effective amount of at least one of the compounds according to the invention.

The compounds according to the invention can be employed alone or, if required, in combination with other active ingredients. The present invention therefore further relates to medicaments comprising at least one of the compounds according to the invention and one or more further active ingredients, in particular for the treatment and/or prophylaxis of the aforementioned disorders.

Suitable combination active ingredients which may be mentioned by way of example and preferably are: lipid metabolism-altering active ingredients, antidiabetics, blood pressure-reducing agents, agents which promote blood flow and/or have antithrombotic effects, antiarrhythmics, antioxidants, chemokine receptor antagonists, p38 kinase inhibitors, NPY agonists, orexin agonists, anorectic agents, PAF-AH inhibitors, anti-inflammatory agents (COX inhibitors, LTB₄ receptor antagonists), and analgesics such as, for example, aspirin.

The present invention relates in particular to combinations of at least one of the compounds according to the invention with at least one lipid metabolism-altering active ingredient, antidiabetic, blood pressure reducing active ingredient, antiarrhythmic and/or agent having antithrombotic effects.

The compounds according to the invention can preferably be combined with one or more

lipid metabolism-altering active ingredients, by way of example and preferably from the group of HMG-CoA reductase inhibitors, inhibitors of HMG-CoA reductase expression, squalene synthesis inhibitors, ACAT inhibitors, LDL receptor inducers, cholesterol absorption inhibitors, polymeric bile acid adsorbents, bile acid reabsorption inhibitors, MTP inhibitors, lipase inhibitors, LPL activators, fibrates, niacin, CETP inhibitors, PPAR-α, PPAR-γ and/or PPAR-δ agonists, RXR modulators, FXR modulators, LXR modulators, thyroid hormones and/or thyroid mimetics, ATP-citrate lyase inhibitors, Lp(a) antagonists, cannabinoid receptor 1 antagonists, leptin receptor agonists, bombesin receptor agonists, histamine receptor agonists, and of antioxidants/radical scavengers;

antidiabetics which are mentioned in the Rote Liste 2004/II, Chapter 12, and, by way of example and preferably, those from the group of sulfonylureas, biguanides, meglitinide derivatives, glucosidase inhibitors, inhibitors of dipeptidyl-peptidase IV (DPP-IV inhibitors), oxadiazolidinones, thiazolidinediones, GLP 1 receptor agonists, glucagon antagonists, insulin sensitizers, CCK 1 receptor agonists, leptin receptor agonists, inhibitors of hepatic enzymes involved in the stimulation of gluconeogenesis and/or glycogenolysis, modulators of glucose uptake, and of potassium channel openers such as, for example, those disclosed in WO 97/26265 and WO 99/03861; blood pressure-reducing active ingredients, by way of example and preferably from the group of calcium antagonists, angiotensin AII antagonists, ACE inhibitors, rennin inhibitors, beta-adrenoceptor antagonists, alpha-adrenoceptor antagonists, diuretics, aldosterone antagonists, mineralocorticoid receptor antagonists, ECE inhibitors, and of vasopeptidase inhibitors;

agents having antithrombotic effects, by way of example and preferably from the group of platelet aggregation inhibitors or of anticoagulants;

antiarrhythmics, especially those for the treatment of supraventricular arrhythmias and tachycardias;

substances for the prophylaxis and treatment of ischemic and reperfusion damage; vasopressin receptor antagonists;

organic nitrates and NO donors;

compounds with positive inotropic activity;

compounds which inhibit the degradation of cyclic guanosine monophosphate (cGMP) and/or cyclic adenosine monophosphate (cAMP), such as, for example, inhibitors of phosphodiesterases (PDE) 1, 2, 3, 4 and/or 5, especially PDE 5 inhibitors such as sildenafil, vardenafil and tadalafil, and PDE 3 inhibitors such as milrinone; natriuretic peptides such as, for example, atrial natriuretic peptide (ANP, anaritide), B-type natriuretic peptide or brain natriuretic peptide (BNP, nesiritide), C-type natriuretic peptide (CNP) and urodilatin;

agonists of the prostacyclin receptor (IP receptor), such as, for example iloprost, beraprost and cicaprost;

calcium sensitizers such as by way of example and preferably levosimendan; potassium supplements;

NO and heme-independent activators of guanylate cyclase, such as in particular the compounds described in WO 01/19355, WO 01/19776, WO 01/19778, WO 01/19780, WO 02/070462 and WO 02/070510;

NO-independent but heme-dependent stimulators of guanylate cyclase, such as in particular the compounds described in WO 00/06568, WO 00/06569, WO 02/42301 and WO 03/095451;

Inhibitors of human neutrophil elastase (HNE), such as, for example, sivelestat and DX-890 (reltran);

compounds which inhibit the signal transduction cascade, such as, for example, tyrosine kinase inhibitors, especially sorafenib, imatinib, gefitinib and erlotinib;

compounds which influence the energy metabolism of the heart, such as, for example, etomoxir, dichloroacetate, ranolazine and trimetazidine;

analgesics; and/or

substances for the prophylaxis and treatment of nausea and vomiting Lipid metabolism-altering active ingredients preferably mean compounds from the group of HMG-CoA reductase inhibitors, squalene synthesis inhibitors, ACAT inhibitors, choleseterol absorption inhibitors, MTP inhibitors, lipase inhibitors, thyroid hormones and/or thyroid mimetics, niacin receptor agonists, CETP inhibitors, PPAR-α agonists, PPAR-γ agonists, PPAR-δ agonists; polymeric bile acid adsorbents, bile acid reabsorption inhibitors, antioxidants/radical scavengers, and cannabinoid receptor 1 antagonists.

In a preferred embodiment of the invention, the compounds according to the invention are administered in combination with an HMG-CoA reductase inhibitor from the class of statins, such as by way of example and preferably lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor, such as by way of example and preferably BMS-188494 or TAK-475.

In a preferred embodiment of the invention, the compounds according to the invention are administered in combination with an ACAT inhibitor, such as by way of example and preferably avasimibe, melinamide, pactimibe, eflucimibe or SMP-797.

In a preferred embodiment of the invention, the compounds according to the invention are administered in combination with a cholesterol absorption inhibitor, such as by way of example and preferably ezetimibe, tiqueside or pamaqueside.

In a preferred embodiment of the invention, the compounds according to the invention are administered in combination with an MTP inhibitor, such as by way of example and preferably implitapide, BMS-201038, R-103757 or JTT-130.

In a preferred embodiment of the invention, the compounds according to the invention are administered in combination with a lipase inhibitor, such as by way of example and preferably orlistat.

In a preferred embodiment of the invention, the compounds according to the invention are administered in combination with a thyroid hormone and/or thyroid mimetic, such as by way of example and preferably D-thyroxine or 3,5,3′-triiodothyronine (T3).

In a preferred embodiment of the invention, the compounds according to the invention are administered in combination with an agonist of the niacin receptor, such as by way of example and preferably niacin, acipimox, acifran or radecol.

In a preferred embodiment of the invention, the compounds according to the invention are administered in combination with a CETP inhibitor, such as by way of example and preferably torcetrapib, JTT-705, BAY 60-5521, BAY 78-7499 or CETP vaccine (Avant). In a preferred embodiment of the invention, the compounds according to the invention are administered in combination with a PPAR-γ agonist, such as by way of example and preferably pioglitazone or rosiglitazone.

In a preferred embodiment of the invention, the compounds according to the invention are administered in combination with a PPAR-δ agonist, such as by way of example and preferably GW-501516 or BAY 68-5042.

In a preferred embodiment of the invention, the compounds according to the invention are administered in combination with a polymeric bile acid adsorbent, such as by way of example and preferably cholestyramine, colestipol, colesolvam, CholestaGel or colestimide.

In a preferred embodiment of the invention, the compounds according to the invention are administered in combination with a bile acid reabsorption inhibitor, such as by way of example and preferably ASBT (=IBAT) inhibitors, such as, for example, AZD-7806, S-8921, AK-105, BARI-1741, SC-435 or SC-635.

In a preferred embodiment of the invention, the compounds according to the invention are administered in combination with an antioxidant/radical scavenger, such as by way of example and preferably probucol, AGI-1067, BO-653 or AEOL-10150.

In a preferred embodiment of the invention, the compounds according to the invention are administered in combination with a cannabinoid receptor 1 antagonist, such as by way of example and preferably rimonabant or SR-147778.

Antidiabetics preferably mean insulin and insulin derivatives, and orally active hypoglycemic active ingredients. Insulin and insulin derivatives includes in this connection both insulins of animal, human or biotechnological origin and mixtures thereof. The orally active hypoglycemic active ingredients preferably include sulfonylureas, biguanides, meglitinide derivatives, glucosidase inhibitors, DPP-IV inhibitors and PPAR-γ agonists.

In a preferred embodiment of the invention, the compounds according to the invention are administered in combination with insulin.

In a preferred embodiment of the invention, the compounds according to the invention are administered in combination with a sulfonylurea, such as by way of example and preferably tolbutamide, glibenclamide, glimepiride, glipizide or gliclazide.

In a preferred embodiment of the invention, the compounds according to the invention are administered in combination with a biguanide, such as by way of example and preferably metformin.

In a preferred embodiment of the invention, the compounds according to the invention are administered in combination with a meglitinide derivative, such as by way of example and preferably repaglinide or nateglinide.

In a preferred embodiment of the invention, the compounds according to the invention are administered in combination with a glucosidase inhibitor, such as by way of example and preferably miglitol or acarbose.

In a preferred embodiment of the invention, the compounds according to the invention are administered in combination with a DPP-IV inhibitor, such as by way of example and preferably sitagliptin or vildagliptin.

In a preferred embodiment of the invention, the compounds according to the invention are administered in combination with a PPAR-γ agonist, for example from the class of thiazolidinediones, such as by way of example and preferably pioglitazone or rosiglitazone.

Blood pressure-reducing agents preferably mean compounds from the group of calcium antagonists, angiotensin AII antagonists, ACE inhibitors, renin inhibitors, beta-adrenoceptor antagonists, alpha-adrenoceptor antagonists and diuretics.

In a preferred embodiment of the invention, the compounds according to the invention are administered in combination with a calcium antagonist, such as by way of example and preferably nifedipine, amlodipine, verapamil or diltiazem.

In a preferred embodiment of the invention, the compounds according to the invention are administered in combination with an angiotensin AII antagonist, such as by way of example and preferably losartan, valsartan, candesartan, embusartan, olmesartan or telmisartan.

In a preferred embodiment of the invention, the compounds according to the invention are administered in combination with an ACE inhibitor, such as by way of example and preferably enalapril, captopril, lisinopril, ramipril, delapril, fosinopril, quinopril, perindopril or trandopril.

In a preferred embodiment of the invention, the compounds according to the invention are administered in combination with a renin inhibitor, such as by way of example and preferably aliskiren, SPP-600 or SPP-800.

In a preferred embodiment of the invention, the compounds according to the invention are administered in combination with a beta-adrenoceptor antagonist, such as by way of example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an alpha-adrenoceptor antagonist, such as by way of example and preferably prazosin.

In a preferred embodiment of the invention, the compounds according to the invention are administered in combination with a diuretic, such as by way of example and preferably furosemide, bumetanide, torsemide, bendroflumethiazide, chlorthiazide, hydrochlorthiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

In a preferred embodiment of the invention, the compounds according to the invention are administered in combination with an aldosterone or mineralocorticoid receptor antagonist, such as by way of example and preferably spironolactone or eplerenone.

In a preferred embodiment of the invention, the compounds according to the invention are administered in combination with a vasopressin receptor antagonist, such as by way of example and preferably conivaptan, tolvaptan, lixivaptan or SR-121463.

In a preferred embodiment of the invention, the compounds according to the invention are administered in combination with an organic nitrate or NO donor, such as by way of example and preferably sodium nitroprusside, glycerol nitrate, isosorbide mononitrate, isosorbide dinitrate, molsidomine or SIN-1, or in combination with inhaled NO.

In a preferred embodiment of the invention, the compounds according to the invention are administered in combination with a compound having positive inotropic activity, such as by way of example and preferably cardiac glycosides (digoxin) and beta-adrenergic and dopaminergic agonists such as isoproterenol, adrenaline, noradrenaline, dopamine or dobutamine.

In a preferred embodiment of the invention, the compounds according to the invention are administered in combination with antisympathotonics such as reserpine, clonidine or alpha-methyldopa, or in combination with potassium channel agonists such as minoxidil, diazoxide, dihydralazine or hydralazine.

Agents having an antithrombotic effect preferably mean compounds from the group of platelet aggregation inhibitors or of anticoagulants.

In a preferred embodiment of the invention, the compounds according to the invention are administered in combination with a platelet aggregation inhibitor, such as by way of example and preferably aspirin, clopidogrel, ticlopidine or dipyridamole.

In a preferred embodiment of the invention, the compounds according to the invention are administered in combination with a thrombin inhibitor, such as by way of example and preferably ximelagatran, melagatran, bivalirudin or clexane.

In a preferred embodiment of the invention, the compounds according to the invention are administered in combination with a GPIIb/IIIa antagonist, such as by way of example and preferably tirofiban or abciximab.

In a preferred embodiment of the invention, the compounds according to the invention are administered in combination with a factor Xa inhibitor, such as by way of example and preferably rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428. In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, such as by way of example and preferably coumarin.

Antiarrhythmics preferably means substances from the group of class la antiarrhythmics (e.g. quinidine), of class Ic antiarrhythmics (e.g. flecainide, propafenone), of class II antiarrhythmics (e.g. metoprolol, atenolol, sotalol, oxprenolol and other beta-receptor blockers), of class III antiarrhythmics (e.g. sotalol, amiodarone) and of class IV antiarrhythmics (e.g. digoxin, and verapamil, diltiazem and other calcium antagonists). Particular preference is given in the context of the present invention to combinations comprising at least one of the compounds according to the invention and one or more further active ingredients selected from the group consisting of HMG-CoA reductase inhibitors (statins), diuretics, beta-adrenoceptor antagonists, alpha-adrenoceptor antagonists, organic nitrates and NO donors, calcium antagonists, ACE inhibitors, angiotensin AII antagonists, aldosterone and mineralocorticoid receptor antagonists, vasopressin receptor antagonists, platelet aggregation inhibitors, anticoagulants and antiarrhythmics, and to the use thereof for the treatment and/or prophylaxis of the aforementioned disorders.

The present invention further relates to medicaments which comprise at least one compound according to the invention, normally together with one or more inert, non-toxic, pharmaceutically suitable excipients, and to the use thereof for the aforementioned purposes.

The compounds according to the invention can act systemically and/or locally. For this purpose, they can be administered in a suitable way such as, for example, by the oral, parenteral, pulmonary, nasal, sublingual, lingual, buccal, rectal, dermal, transdermal, conjunctival or otic route or as an implant or stent. The compounds according to the invention can be administered in administration forms suitable for these administration routes.

Suitable for oral administration are administration forms which function according to the prior art and deliver the compounds according to the invention rapidly and/or in modified fashion, and which contain the compounds according to the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions. Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation (inter alia powder inhalers, nebulizers), nasal drops, solutions or sprays, tablets for lingual, sublingual or buccal administration, films/wafers or capsules, suppositories, preparations for the eyes or ears, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

Oral or parenteral administration is preferred, especially oral and intravenous administration.

The compounds according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and masking flavors and/or odors. It has generally proved advantageous to administer on parenteral administration amounts of about 0.001 to 1 mg/kg, preferably about 0.01 to 0.5 mg/kg, of body weight to achieve effective results, and on oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. EXAMPLES

Abbreviations and acronyms:
Ac Acetyl
aq. aqueous, aqueous solution
Boc Tert-Butoxycarbonyl
conc. concentrated
DMAP 4-N,N-Dimethylaminopyridine
DMF N,N-Dimethylformamide
DMSO Dimethyl sulfoxide
EDC 1-(3-Dimethylaminopropyl)-3-ethylcarbodiimide hydrochloride
ESI Electrospray ionization (in MS)
EtOH Ethanol
h Hour(s)
HOAc Acetic acid
HPLC High pressure, high performance liquid chromatography
LC-MS Coupled liquid chromatography-mass spectrometry
min Minute(s)
MS Mass spectrometry
NMM N-Methylmorpholine
NMR Nuclear magnetic resonance spectrometry
p para
quant. quantitative (for yield)
RT Room temperature
Rₜ Retention time (in HPLC)
sat. saturated
tert. Tertiary
TFA Trifluoroacetic acid
THF Tetrahydrofuran
UV Ultraviolet spectrometry
v/v Volume to volume ratio (of a solution)
Z Benzyloxycarbonyl

LC-MS methods:

Method 1:

MS instrument type: Micromass ZQ; HPLC instrument type: Waters Alliance 2795; column: Merck Chromolith SpeedROD RP-18e 50 mm×4.6 mm; eluent A: 1 I water+0.5 ml 50% formic acid, eluent B: 1 l acetonitrile+0.5 ml 50% formic acid; gradient: 0.0 min 10% B→3.0 min 95% B→4.0 min 95% B; flow rate: 0.0 min 1.0 ml/min→3.0 min 3.0 ml/min+4.0 min 3.0 ml/min; oven: 35° C.; UV detection: 210 nm.

Method 2:

Instrument: Micromass Platform LCZ with HPLC Agilent Series 1100; column: Thermo Hypersil GOLD 3μ, 20 mm×4 mm; eluent A: 1 l water+0.5 ml 50% formic acid, eluent B: 1 I acetonitrile+0.5 ml 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 3:

MS instrument type: Waters ZQ; HPLC instrument type: Waters Alliance 2795; column: Phenomenex Onyx Monolithic C18, 100 mm×3 mm; eluent A: 1 l water+0.5 ml 50% formic acid, eluent B: 1 l acetonitrile+0.5 ml 50% formic acid; gradient: 0.0 min 90% A→2 min 65% A→4.5 min 5% A -4 6 min 5% A; flow rate: 2 ml/min; oven: 40° C.; UV detection: 210 nm.

Method 4:

Instrument: Micromass Quattro LCZ with HPLC Agilent Series 1100; UV DAD; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

Method 5:

Instrument: Micromass Quattro Premier with Waters UPLC Acquity; column: Thermo Hypersil GOLD 1.9μ, 50 mm×1 mm; eluent A: 1 l water+0.5 ml 50% formic acid, eluent B: 1 l acetonitrile+0.5 ml 50% formic acid; gradient: 0.0 min 90% A→0.1 min 90% A→1.5 min 10% A→2.2 min 10% A; flow rate: 0.33 ml/min; oven: 50° C.; UV detection: 210 nm.

Method 6:

MS instrument type: Micromass ZQ; HPLC instrument type: Waters Alliance 2795; column: Phenomenex Synergi 2.5μ MAX-RP 100A Mercury 20 mm×4 mm; eluent A: 1 l water+0.5 ml 50% formic acid, eluent B: 1 l acetonitrile+0.5 ml 50% formic acid; gradient: 0.0 min 90% A→0.1 min 90% A→3.0 min 5% A ->4.0 min 5% A→90% A; flow rate: 2 ml/min; oven: 50° C.; UV detection: 210 nm.

Method 7:

MS instrument type: Micromass ZQ; HPLC instrument type: HP 1100 Series; UV DAD; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 8:

Instrument: Waters Acquity SQD UPLC system; column: Waters Acquity UPLC HSS T3 1.8μ 50 mm×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; flow rate: 0.40 ml/min; oven: 50° C.; UV detection: 210-400 nm.

Method 9:

MS instrument type: M-40 DCI (NH₃); HPLC instrument type: HP 1100 with DAD detection; column: Kromasil 100 RP-18, 60 mm×2.1 mm, 3.5 μm; eluent A: 5 ml HClO₄ (70%)/liter water, eluent B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

Method 10:

Instrument: Micromass Quattro Micro MS with HPLC Agilent series 1100; column: Thermo Hypersil GOLD 3μ 20 mm×4 mm; eluent A: 1 l water+0.5 ml 50% formic acid, eluent B: 1 l acetonitrile+0.5 ml 50% formic acid; gradient: 0.0 min 100% A→3.0 min 10% A→4.0 min 10% A→4.01 min 100% A (flow rate 2.5 ml/min)→5.00 min 100% A; oven: 50° C.; flow rate: 2 ml/min; UV detection: 210 nm.

Method 11:

Instrument: Micromass Quattro LCZ with HPLC Agilent Series 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

Method 12:

MS instrument type: Waters ZQ; HPLC instrument type: Waters Alliance 2795; column: Merck Chromolith RP-18e, 100 mm×3 mm; eluent A: 1 l water+0.5 ml 50% formic acid, eluent B: 1 l acetonitrile+0.5 ml 50% formic acid; gradient: 0.0 min 90% A→2 min 65% A→4.5 min 5% A→6 min 5% A; flow rate: 2 ml/min; oven: 40° C.; UV detection: 210 nm.

Starting compounds and intermediates:

### Example 1A

### 4-{[(4S)-2,2-Dimethyl-1,3-dioxolan-4-yl]methoxy}benzaldehyde

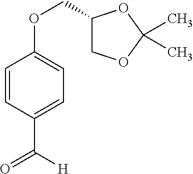

An amount of 12.5 g (102.4 mmol) of 4-hydroxybenzaldehyde were introduced under argon in 166 ml of dry DMF and admixed at RT with 42.4 g (307.1 mmol) of potassium carbonate and also 20.05 g (133.1 mmol) of (R)-(−)-3-chloro-1,2-propanediol acetonide. The batch was stirred at 160° C. for 16 hours. The batch was then admixed with water and extracted twice with ethyl acetate. The combined organic phases were washed with saturated aqueous sodium chloride solution and dried over magnesium sulfate. Following filtration, the solvent was removed on a rotary evaporator and the residue was purified by means of column chromatography on silica gel (eluent: cyclohexane/ethyl acetate 10:2).

Yield: 20.0 g (82% of theory)

¹H-NMR (400 MHz, DMSO-d₆): δ=9.89 (s, 1H), 7.85 (d, 2H), 7.03 (d, 2H), 4.50 (q, 1H), 4.22-4.09 (m, 2H), 4.04 (dd, 1H), 3.92 (dd, 1H), 1.48 (s, 3H), 1.41 (s, 3H).

LC-MS (method 9): Rₜ=4.02 min; MS (ESIpos): m/z=254 [M+NH₄]⁺.

### Example 2A

### 4-{[(4R)-2,2-Dimethyl-1,3-dioxolan-4-yl]methoxy}benzaldehyde

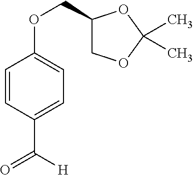

An amount of 31.2 g (255.4 mmol) of 4-hydroxybenzaldehyde was introduced in 400 ml of dry DMF and admixed at RT with 105.7 g (766.1 mmol) of potassium carbonate and also 50.0 g (332.0 mmol) of (S)-(−)-3-chloro-1,2-propanediol acetonide. The batch was stirred at 160° C. for 16 hours. The batch was then admixed with 4000 ml of water and extracted with three times 500 ml of ethyl acetate. The combined organic phases were washed once each with 500 ml of water and 500 ml of saturated aqueous sodium chloride solution. After drying over magnesium sulfate, the solvent was removed on a rotary evaporator and the residue was purified by column chromatography on silica gel 60 (eluent gradient: ethyl acetate/petroleum ether 1:9→2:8).

Yield: 40.4 g (63% of theory)

¹H-NMR (400 MHz, DMSO-d₆): δ=9.90 (s, 1H), 7.85 (d, 2H), 7.03 (d, 2H), 4.50 (q, 1H), 4.22-4.09 (m, 2H), 4.04 (dd, 1H), 3.92 (dd, 1H), 1.48 (s, 3H), 1.41 (s, 3H).

LC-MS (method 9): Rₜ=3.97 min; MS (ESIpos): m/z=254 M+NH₄]⁺.

### Example 3A

### 2-Amino-4-(4-{[(4S)-2,2-dimethyl-1,3-dioxolan-4-yl]methoxy}phenyl)-6-mercaptopyridine-3,5-dicarbonitrile

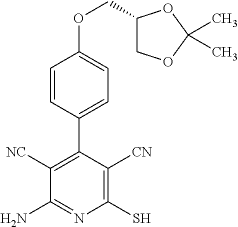

An amount of 44.0 g (186.2 mmol) of the compound from example 1A and 37.3 g (372.5 mmol) of cyanothioacetamide were introduced in 800 ml of ethanol. The reaction mixture was admixed at room temperature with 37.6 g (372.5 mmol) of 4-methylmorpholine and heated at reflux with stirring for 3 hours. After cooling to RT, it was stirred at this temperature for a further 16 hours. The precipitate was isolated by suction filtration, washed with ethanol and dried under reduced pressure. The product was used without further purification in the subsequent reaction.

Yield: 22.8 g (32% of theory)

¹H-NMR (400 MHz, DMSO-d₆): δ=7.69-7.37 (br. s, 2H), 7.42 (d, 2H), 7.10 (d, 2H), 4.48-4.39 (m, 1H), 4.15-4.02 (m, 2H), 3.78 (dd, 1H), 3.66 (dd, 1H), 2.77-2.68 (br. s, 1H), 1.37 (s, 3H), 1.31 (s, 3H).

LC-MS (method 1): Rₜ=1.75 min; MS (ESIpos): m/z=383 [M+H]⁺.

### Example 4A

### 2-Amino-4-(4-{[(4R)-2,2-dimethyl-1,3-dioxolan-4-yl]methoxy}phenyl)-6-mercaptopyridine-3,5-dicarbonitrile

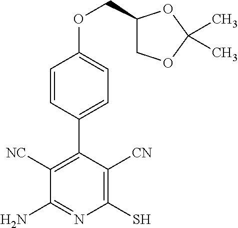

An amount of 40.4 g (171.0 mmol) of the compound from example 2A and 34.2 g (342.0 mmol) of cyanothioacetamide were introduced in 700 ml of ethanol. The reaction mixture was admixed with 34.5 g (342.0 mmol) of 4-methylmorpholine and heated at reflux with stirring for 3 hours. After cooling to RT, it was stirred at this temperature for a further 16 hours. The precipitate was isolated by suction filtration, washed with around 100 ml of ethanol and dried in a drying cabinet. The product was used without further Purification in the subsequent reaction.

Yield: 19.5 g (29% of theory)

¹H-NMR (400 MHz, DMSO-d₆): δ=7.63-7.31 (br. s, 2H), 7.41 (d, 2H), 7.09 (d, 2H), 4.49-4.38 (m, 1H), 4.15-3.99 (m, 2H), 3.78 (dd, 1H), 3.66 (dd, 1H), 2.77-2.68 (br. s, 1H), 1.37 (s, 3H), 1.32 (s, 3H).

LC-MS (method 11): Rₜ=1.95 min; MS (ESIpos): m/z=424 [M+H+CH₃CN]⁺.

### Example 5A

### 4-(Chloromethyl)-2-(4-chlorophenyl)-1,3-oxazole

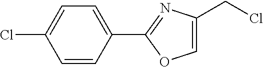

An amount of 123.8 g (795.5 mmol) of 4-chlorobenzenecarboxamide and 101.0 g (795.5 mmol) of 1,3-dichloroacetone were stirred at 135° C. for an hour. A melt was formed. The batch was subsequently cooled to RT with stirring, and at this temperature it was admixed cautiously with 200 ml of concentrated sulfuric acid and stirred for 30 minutes. The resulting suspension was poured into ice-water and stirred for a further 30 minutes. The precipitate formed was then isolated by suction filtration, washed with water and purified by flash chromatography on silica gel (eluent: dichloromethane). The solvent was removed on a rotary evaporator and the residue was dried under reduced pressure. This gave 95.5 g (53% of theory) of the target compound.

¹H-NMR (400 MHz, DMSO-d₆): δ=8.30 (s, 1H), 7.99 (d, 2H), 7.62 (d, 2H), 4.75 (s, 2H).

LC-MS (method 2): Rₜ=3.78 min; MS (ESIpos): m/z=228 [M+H]⁺.

### Example 6A

### 2-Amino-6-({[2-(4-chlorophenyl)-1,3-oxazol-4-yl]methyl}sulfanyl)-4-(4-{[(4S)-2,2-dimethyl-1,3-dioxolan-4-yl]methoxy}phenyl)pyridine-3,5-dicarbonitrile

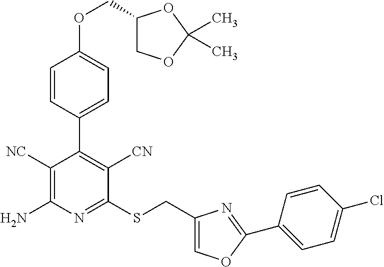

An amount of 150 mg (0.39 mmol) of the compound from example 3A and 98 mg (0.43 mmol) of the compound from example 5A were suspended together with 99 mg (1.18 mmol) of sodium hydrogencarbonate in 2 ml of dry DMF. The reaction mixture was stirred at RT for 20 hours. The batch was thereafter purified directly by means of preparative HPLC (column: YMC GEL ODS-AQ S-5/15 μm; eluent gradient: acetonitrile/water 10:90→95:5).

Yield: 147 mg (65% of theory)

¹H-NMR (400 MHz, DMSO-d₆): δ=8.37 (s, 1H), 8.29-7.91 (br. s, 2H), 7.97 (d, 2H), 7.61 (d, 2H), 7.47 (d, 2H), 7.12 (d, 2H), 4.48-4.39 (m, 1H), 4.42 (s, 2H), 4.16-4.03 (m, 3H), 3.77 (dd, 1H), 1.37 (s, 3H), 1.31 (s, 3H).

LC-MS (method 3): Rₜ=4.23 min; MS (ESIpos): m/z=574 [M+H]⁺.

### Example 7A

### 2-Amino-6-({[2-(4-chlorophenyl)-1,3-oxazol-4-yl]methyl}sulfanyl)-4-(4-{[4R)-2,2-dimethyl-1,3-dioxolan-4-yl]methoxy}phenyl)pyridine-3,5-dicarbonitrile

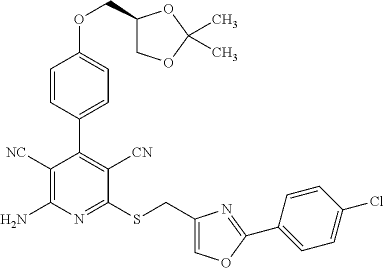

An amount of 70 mg (0.18 mmol) of the compound from example 4A and 46 mg (0.20 mmol) of the compound from example 5A were suspended together with 46 mg (0.55 mmol) of sodium hydrogencarbonate in 1.9 ml of dry DMF. The reaction mixture was stirred at RT for 20 hours. The batch was subsequently freed from the solvent on a rotary evaporator and the residue was purified by preparative HPLC (column: YMC GEL ODS-AQ S-5/15 μm; eluent gradient: acetonitrile/water 10:90→95:5).

Yield: 79 mg (75% of theory)

¹H-NMR (400 MHz, DMSO-d₆): δ=8.37 (s, 1H), 8.30-8.01 (br. s, 2H), 7.97 (d, 2H), 7.60 (d, 2H), 7.48 (d, 2H), 7.12 (d, 2H), 4.48-4.40 (m, 1H), 4.42 (s, 2H), 4.16-4.03 (m, 3H), 3.78 (dd, 1H), 1.37 (s, 3H), 1.31 (s, 3H).

LC-MS (method 7): Rₜ=2.99 min; MS (ESIpos): m/z=574 [M+H]⁺.

### Example 8A

### 2-Amino-6-({[2-(4-chlorophenyl)-1,3-oxazol-4-yl]methyl}sulfanyl)-4-(4-{[(2R)-2,3-dihydroxypropyl]oxy}phenyl)pyridine-3,5-dicarbonitrile

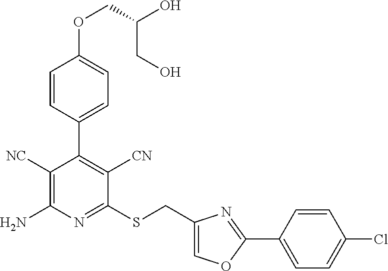

An amount of 127.1 g (221.4 mmol) of the compound from example 6A were suspended in 800 ml of ethanol and admixed with 800 ml of 37% strength hydrochloric acid. The mixture was stirred under reflux overnight. After cooling to room temperature, the precipitate formed was isolated by suction filtration, washed with ethanol and dried under reduced pressure at 50° C. overnight. This gave 108.3 g (92% of theory) of the target compound.

¹H-NMR (400 MHz, DMSO-d₆): δ=8.37 (s, 1H), 8.30-7.89 (br. s, 2H), 7.98 (d, 2H), 7.61 (d, 2H), 7.48 (d, 2H), 7.10 (d, 2H), 5.00 (d, 1H), 4.70 (t, 1H), 4.42 (s, 2H), 4.09 (dd, 1H), 3.98-3.92 (m, 1H), 3.81 (q, 1H), 3.50-3.43 (m, 2H).

LC-MS (method 4): Rₜ=2.51 min; MS (ESIpos): m/z=534 [M+H]⁺.

### Example 9A

### 2-Amino-6-({[2-(4-chlorophenyl)-1,3-oxazol-4-yl]methyl}sulfanyl)-4-(4-{[(2S)-2,3-dihydroxypropyl]oxy}phenyl)pyridine-3,5-dicarbonitrile

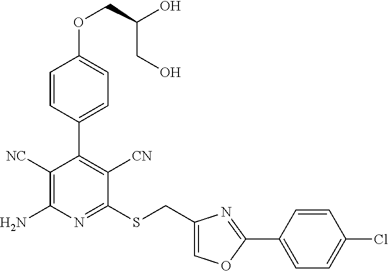

An amount of 400 mg (0.70 mmol) of the compound from example 7A was introduced in 17 ml of acetic acid and then admixed cautiously with 8.6 ml of water. The batch was stirred at RT for 12 hours. After the reaction mixture had been concentrated on a rotary evaporator, the residue was purified directly by preparative HPLC (column: YMC GEL ODS-AQ S-5/15 μm; eluent gradient: acetonitrile/water 10:90→95:5). Removal of the solvent on a rotary evaporator gave the product as a white solid.

Yield: 340 mg (91% of theory)

¹H-NMR (400 MHz, DMSO-d₆): δ=8.37 (s, 1H), 8.27-7.91 (br. s, 2H), 7.98 (d, 2H), 7.60 (d, 2H), 7.47 (d, 2H), 7.10 (d, 2H), 5.00 (d, 1H), 4.70 (t, 1H), 4.42 (s, 2H), 4.09 (dd, 1H), 3.96 (dd, 1H), 3.70 (q, 1H), 3.46 (t, 2H).

LC-MS (method 7): Rₜ=2.48 min; MS (ESIpos): m/z=534 [M+H]⁺.

### Example 10A

### (2S)-3-{4-[2-Amino-6-({[2-(4-chlorophenyl)-1,3-oxazol-4-yl]methyl}sulfanyl)-3,5-dicyanopyridin-4-yl]phenoxy}propane-1,2-diyl(2S,2′S)-bis{2-[(tert-butoxycarbonyl)amino]propanoate}

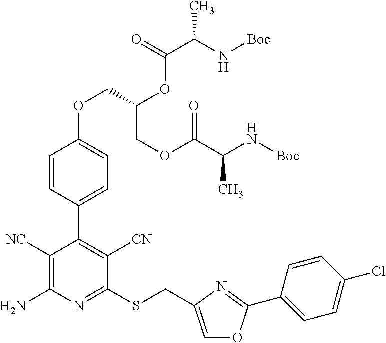

An amount of 5 g (9.36 mmol) of the compound from example 8A, 7.09 g (37.45 mmol) of N-Boc-L-alanine, 8.975 g (46.82 mmol) of 1-(3-dimethylaminopropyl)-3-ethylcarbodiimide hydrochloride and 1.144 g (9.36 mmol) of 4-N,N-dimethylaminopyridine were combined in 500 ml of dichloromethane and treated in an ultrasound bath for 30 minutes. The batch was subsequently shaken with 10% strength citric acid solution and thereafter with 10% strength sodium hydrogencarbonate solution until N-Boc-L-alanine was no longer detectable in the organic phase. The organic phase was then dried over magnesium sulfate and concentrated under reduced pressure. The residue was taken up in dichloromethane and admixed with diethyl ether. The precipitate formed was isolated by suction filtration. Drying of the solid left 6.01 g (73% of theory) of the target compound.

¹H-NMR (400 MHz, DMSO-d₆): δ=8.36 (s, 1H), 8.33-8.02 (br. m, 2H), 7.97 (d, 2H), 7.60 (d, 2H), 7.48 (d, 2H), 7.30 (m, 2H), 7.12 (d, 2H), 5.34 (m, 1H), 4.42 (s, 2H), 4.38-4.21 (m, 4H), 4.03 (m, 2H), 1.36 (s, 18H), 1.26-1.22 (m, 6H).

LC-MS (Method 5): Rₜ=1.61 min; MS (ESIpos): m/z=876 [M+H]⁺.

### Example 11A

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis{[(tert-butoxycarbonyl)amino]acetate}

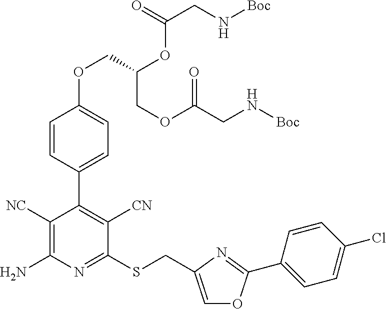

An amount of 300 mg (0.562 mmol) of the compound from Example 8A was introduced in 20 ml of dichloromethane and admixed with 217 mg (1.236 mmol) of N-(tert-butoxycarbonyl)glycine, 237 mg (1.236 mmol) of 1-(3-dimethylaminopropyl)-3-ethylcarbodiimide hydrochloride and 7 mg (0.056 mmol) of 4-N,N-dimethylaminopyridine. The mixture was stirred at room temperature overnight. The solvent was thereafter stripped off under reduced pressure and the crude product was purified directly by means of preparative HPLC. This gave 448 mg (94% of theory) of the target compound.

LC-MS (Method 7): Rₜ=3.07 min; MS (ESIpos): m/z=848 [M+H]⁺.

### Example 12A

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis{5-[(tert-butoxycarbonyl)amino]pentanoate}

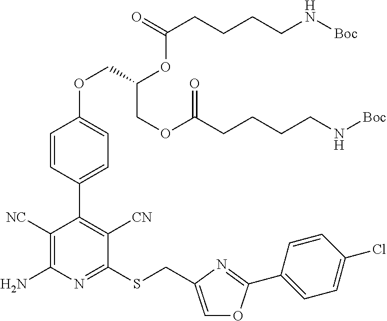

An amount of 150 mg (0.281 mmol) of the compound from Example 8A was introduced in 10 ml of dichloromethane and admixed with 183 mg (0.843 mmol) of 5-[(tert-butoxycarbonyl)amino]pentanoic acid, 162 mg (0.843 mmol) of 1-(3-dimethylaminopropyl)-3-ethylcarbodiimide hydrochloride and 3.4 mg (0.028 mmol) of 4-N,N-dimethylaminopyridine. The mixture was stirred at room temperature overnight. The solvent was thereafter stripped off under reduced pressure and the crude product was purified directly by means of preparative HPLC. This gave 201 mg (77% of theory) of the target compound.

LC-MS (Method 10): Rₜ=2.96 min; MS (ESIpos): m/z=932 [M+H]⁺.

### Example 13A

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis{2,5-bis[(tert-butoxycarbonyl)amino]pentanoate}

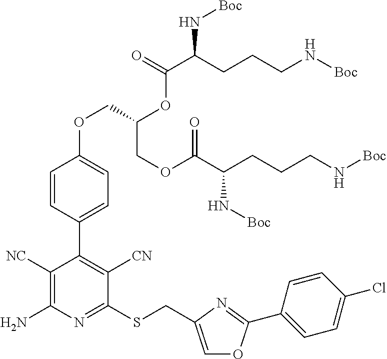

The title compound was prepared in the same way as for the preparation of Example 12A, starting from the compound from Example 8A and commercially available N², N⁵-bis(tert-butoxycarbonyl)-L-ornithine.

Yield: 85% of theory.

LC-MS (Method 10): Rₜ=3.11 min; MS (ESIpos): m/z=1162 [M+H]⁺.

### Example 14A

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis{2,4-bis[(tert-butoxycarbonyl)amino]butanoate}

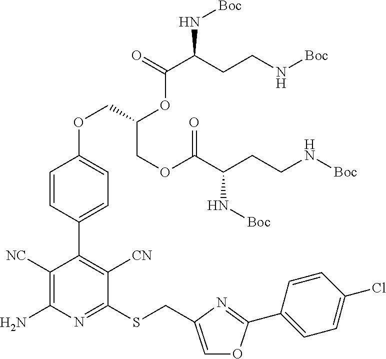

An amount of 200 mg (0.375 mmol) of the compound from Example 8A was introduced in 15 ml of dichloromethane and admixed with 412 mg (0.824 mmol) of the dicyclohexylamine salt of (2S)-2,4-bis[(tert-butoxycarbonyl)amino]butanoic acid, 158 mg (0.824 mmol) of 1-(3-dimethylaminopropyl)-3-ethylcarbodiimide hydrochloride and 4.6 mg (0.037 mmol) of 4-N,N-dimethylaminopyridine. The mixture was stirred at room temperature overnight. Thereafter a further 187 mg (0.375 mmol) of the dicyclohexylamine salt of (2S)-2,4-bis[(tert-butoxycarbonyl)amino]butanoic acid and also 72 mg (0.375 mmol) of 1-(3-dimethylaminopropyl)-3-ethylcarbodiimide hydrochloride were added. After two-hour stirring at room temperature, the solvent was stripped off under reduced pressure and the crude product was purified by means of preparative HPLC. This gave 262 mg (62% of theory) of the target compound. LC-MS (Method 7): Rₜ=3.31 min; MS (ESIpos): m/z=1134 [M+H]⁺.

### Example 15A

### (2S)-3-{4-[2-Amino-6-({[2-(4-chlorophenyl)-1,3-oxazol-4-yl]methyl}sulfanyl)-3,5-dicyanopyridin-4-yl]phenoxy}propane-1,2-diyl bis{3-[(tert-butoxycarbonyl)amino]propanoate}

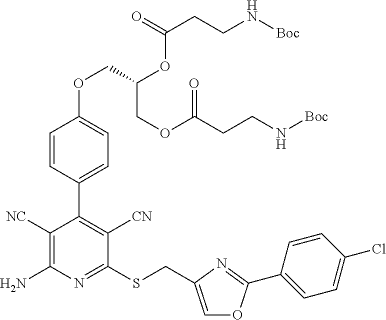

An amount of 200 mg (0.375 mmol) of the compound from example 8A was introduced in 10 ml of dichloromethane/DMF (1:1), admixed with 213 mg (1.124 mmol) of N-(tert-butoxycarbonyl)-β-alanine, 215 mg (1.124 mmol) of 1-(3-dimethylaminopropyl)-3-ethylcarbodiimide hydrochloride and 4.6 mg (0.037 mmol) of 4-N,N-dimethylaminopyridine and stirred at room temperature overnight. The reaction mixture was subsequently purified directly by means of preparative HPLC (acetonitrile/water gradient 10:90→95:5). This gave 126 mg (38% of theory) of the target compound.

LC-MS (Method 6): Rₜ=2.67 min; MS (ESIpos): m/z=876 [M+H]⁺.

### Example 16A

### (2R)-3-{40[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis{2,5-bis[(tert-butoxycarbonyl)amino]pentanoate}

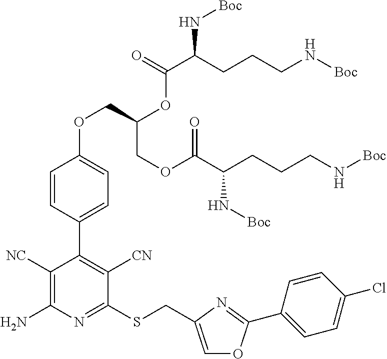

An amount of 374 mg (1.124 mmol) of N²,N⁵-bis(tert-butoxycarbonyl)-L-ornithine was introduced in 3 ml of DMF and admixed initially with 93 mg (0.487 mmol) of 1-(3-dimethylaminopropyl)-3-ethylcarbodiimide hydrochloride and 23 mg (0.187 mmol) of 4-N,N-dimethylaminopyridine, before 200 mg (0.375 mmol) of the compound from Example 9A were added. The mixture was stirred at room temperature overnight. Thereafter a further 93 mg (0.487 mmol) of 1-(3-dimethylaminopropyl)-3-ethylcarbodiimide hydrochloride and 23 mg (0.187 mmol) of 4-N,N-dimethylaminopyridine were added. After three-hour stirring at room temperature, the reaction mixture was purified directly by means of preparative HPLC (acetonitrile/water gradient 10:90→95:5). This gave 344 mg (79% of theory) of the target compound.

LC-MS (Method 6): Rₜ=2.90 min; MS (ESIpos): m/z=1163 [M+H]⁺.

### Example 17A

### (2S)-3-{4-[2-Amino-6-({[2-(4-chlorophenyl)-1,3-oxazol-4-yl]methyl}sulfanyl)-3,5-dicyanopyridin-4-yl]phenoxy}propane-1,2-diyl(2R,2′R)-bis{2-[(tert-butoxycarbonyl)amino]propanoate}

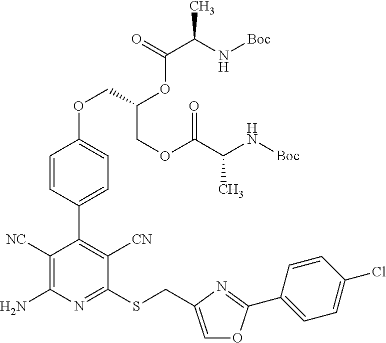

An amount of 1.063 g (5.618 mmol) of N-(tert-butoxycarbonyl)-D-alanine was introduced in 10 ml of DMF and admixed with 448 mg (2.341 mmol) of 1-(3-dimethylaminopropyl)-3-ethylcarbodiimide hydrochloride and 114 mg (0.936 mmol) of 4-N,N-dimethylaminopyridine. After 5 minutes of stirring, 500 mg (0.936 mmol) of the compound from example 8A were added and the mixture was stirred at room temperature for 2 hours. The product was subsequently isolated by means of preparative HPLC (acetonitrile/water gradient 10:90→95:5). This gave 676 mg (82% of theory) of the target compound.

LC-MS (Method 8): Rₜ=1.42 min; MS (ESIneg): m/z=874 [M−H]⁻.

### Example 18A

### (2R)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl-(2S,2′S)bis{2-[(tert-butoxycarbonyl)amino]-3-methylbutanoate}

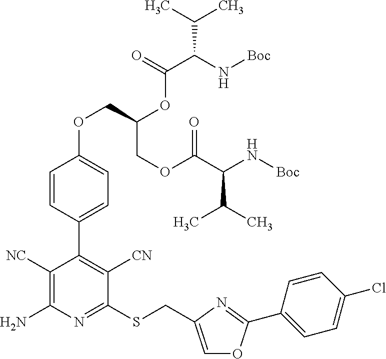

An amount of 16.3 mg (0.075 mmol) of N-(tert-butoxycarbonyl)-L-valine was introduced in 10 ml of dichloromethane and admixed in succession with 15.8 mg (0.082 mmol) of 1-(3-dimethylaminopropyl)-3-ethylcarbodiimide hydrochloride, 0.5 mg (0.004 mmol) of 4-N,N-dimethylaminopyridine and 20 mg (0.037 mmol) of the compound from Example 9A. Subsequently the mixture was stirred at room temperature overnight. The product was thereafter isolated by means of preparative HPLC. This gave 24 mg (69% of theory) of the target compound.

LC-MS (Method 7): Rₜ=3.43 min; MS (ESIneg): m/z=930 [M−H]⁻.

### Example 19A

### (2R)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis{2-[(tert-butoxycarbonyl)amino]propanoate}

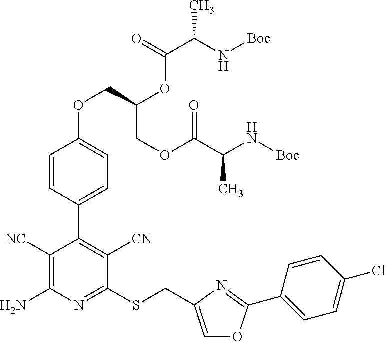

An amount of 53 mg (0.28 mmol) of N-(tert-butoxycarbonyl)-L-alanine was introduced in 37 ml of dichloromethane and admixed in succession with 59 mg (0.309 mmol) of 1-(3-dimethylaminopropyl)-3-ethylcarbodiimide hydrochloride, 1.7 mg (0.014 mmol) of 4-N,N-dimethylaminopyridine and 75 mg (0.14 mmol) of the compound from Example 9A. Subsequently the mixture was stirred at room temperature overnight. The batch was thereafter poured into a mixture of saturated aqueous ammonium chloride solution and ethyl acetate. The organic phase was separated off, dried over magnesium sulphate and concentrated. The crude product was purified by means of preparative HPLC (acetonitrile/water gradient 10:90→95:5). This gave 77 mg (63% of theory) of the target compound.

LC-MS (Method 6): Rₜ=2.71 min; MS (ESIneg): m/z=874 [M−H]⁻.

### Example 20A

### (2R)-3-{4-[2-Amino-6-({([2-(4-chlorophenyl)-1,3-oxazol-4-yl]methyl}sulphanyl)-3,5-dicyanopyridin-4-yl]phenoxy}propane-1,2-diyl bis{4-[(tert-butoxycarbonyl)amino]butanoate}

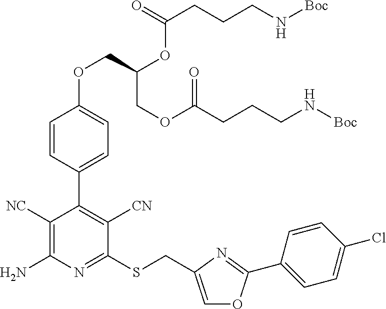

An amount of 57 mg (0.281 mmol) of 4-[(tert-butoxycarbonyl)amino]butanoic acid was introduced in 37 ml of dichloromethane and admixed in succession with 59 mg (0.309 mmol) of 1-(3-dimethylaminopropyl)-3-ethylcarbodiimide hydrochloride, 1.7 mg (0.014 mmol) of 4-N,N-dimethylaminopyridine and 75 mg (0.14 mmol) of the compound from Example 9A. Subsequently the mixture was stirred at room temperature overnight. The batch was thereafter poured into a mixture of saturated aqueous ammonium chloride solution and ethyl acetate. The organic phase was separated off, dried over magnesium sulphate and concentrated. The crude product was purified by means of preparative HPLC (acetonitrile/water gradient 10:90-4 95:5). This gave 78 mg (61% of theory) of the target compound.

LC-MS (Method 5): Rₜ=1.59 min; MS (ESIneg): m/z=903 [M−H]⁻.

### Example 21A

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis(2,6-bis[(tert-butoxycarbonyl)amino]hexanoate}

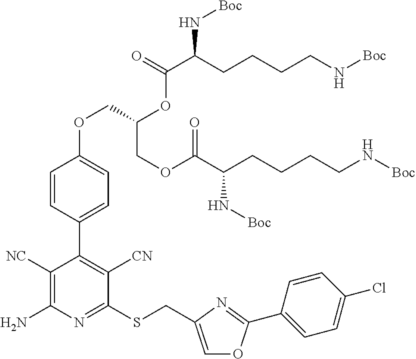

An amount of 649 mg (1.873 mmol) of N²,N⁶-bis(tert-butoxycarbonyl)-L-lysine was introduced in 200 ml of dichloromethane and admixed in succession with 449 mg (2.341 mmol) of 1-(3-dimethylaminopropyl)-3-ethylcarbodiimide hydrochloride, 5.7 mg (0.047 mmol) of 4-N,N-dimethylaminopyridine and 250 mg (0.468 mmol) of the compound from Example 8A. Subsequently the mixture was stirred at room temperature for 5 hours. The batch was thereafter extracted by shaking twice with 10% strength citric acid solution and three times with 10% strength sodium hydrogen carbonate solution. The organic phase was separated off, dried over magnesium sulphate and concentrated. The crude product was purified by means of preparative HPLC. This gave 425 mg (76% of theory) of the target compound.

LC-MS (Method 5): Rₜ=1.76 min; MS (ESIpos): m/z=1190 [M+H]⁺.

### Example 22A

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis{4-[(tert-butoxycarbonyl)amino]butanoate}

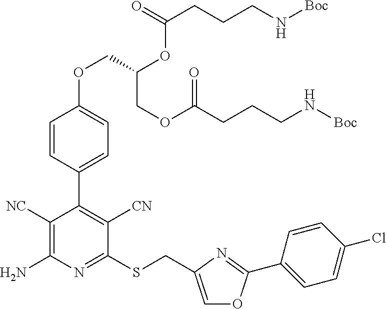

An amount of 342.5 mg (1.685 mmol) of 4-[(tert-butoxycarbonyl)amino]butanoic acid was introduced in 30 ml of dichloromethane and admixed in succession with 323 mg (1.685 mmol) of 1-(3-dimethylaminopropyl)-3-ethylcarbodiimide hydrochloride, 34 mg (0.281 mmol) of 4-N,N-dimethylaminopyridine and 300 mg (0.562 mmol) of the compound from Example 8A. Subsequently the mixture was stirred at room temperature for 2 hours. The batch was thereafter diluted with 100 ml of dichloromethane and extracted by shaking once with 10% strength citric acid solution and three times with 10% strength sodium hydrogencarbonate solution. The organic phase was separated off, dried over magnesium sulphate and concentrated. The crude product was purified by means of preparative HPLC. This gave 290 mg (57% of theory) of the target compound.

LC-MS (Method 7): Rₜ=3.18 min; MS (ESIpos): m/z=904 [M+H]⁺.

### Example 23A

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis{6-[(tert-butoxycarbonyl)amino]hexanoate

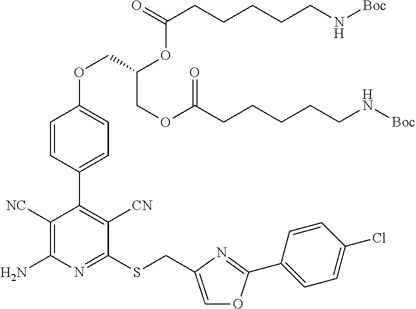

An amount of 390 mg (1.685 mmol) of 6-[(tert-butoxycarbonyl)amino]hexanoic acid was introduced in 30 ml of dichloromethane and admixed in succession with 323 mg (1.685 mmol) of 1-(3-dimethylaminopropyl)-3-ethylcarbodiimide hydrochloride, 34 mg (0.281 mmol) of 4-N,N-dimethylaminopyridin and 300 mg (0.562 mmol) of the compound from Example 8A. Subsequently the mixture was stirred at room temperature for 1 hour. The batch was thereafter diluted with 100 ml of dichloromethane and extracted by shaking once with 10% strength citric acid solution and three times with 10% strength sodium hydrogencarbonate solution. The organic phase was separated off, dried over magnesium sulphate and concentrated. The crude product was purified by means of preparative HPLC. This gave 279 mg (52% of theory) of the target compound.

LC-MS (Method 7): Rₜ=3.30 min; MS (ESIpos): m/z=960 [M+H]⁺.

### Example 24A

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,3R,2′S,3R)-bis{3-tert-butoxy-2-[(tert-butoxycarbonyl)amino]butanoate

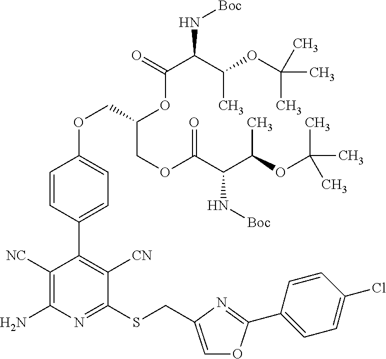

An amount of 1.293 g (2.421 mmol) of the compound from Example 8A was introduced in 26 ml of dichloromethane/DMF (1:1) and admixed in succession with 2.00 g (7.262 mmol) of N-(tert-butoxycarbonyl)-O-tert-butyl-L-threonine, 1.625 g (8.474 mmol) of 1-(3-dimethylaminopropyl)-3-ethylcarbodiimide hydrochloride and 59 mg (0.484 mmol) of 4-N,N-dimethylaminopyridine. The mixture was stirred at RT overnight. Thereafter a further 0.667 g (2.42 mmol) of N-(tert-butoxycarbonyl)-O-tert-butyl-L-threonine was added and the batch was stirred at RT for a further 8 hours. The reaction mixture was then diluted with water and dichloromethane and the phases were separated. The aqueous phase was back-extracted twice with dichloromethane. The combined organic phases were washed once with water, dried over sodium sulphate and concentrated. The crude product was purified by means of column chromatography on silica get (eluent: cyclohexane/ethyl acetate 7:3). This gave 2.17 g (85% of theory) of the target compound.

LC-MS (Method 6): Rₜ=3.29 min; MS (ESIneg): m/z=1047 [M−H]⁻.

### Example 25A

### 2-(4-Chlorophenyl)-4,5-dimethyl-1,3-oxazol 3-oxide

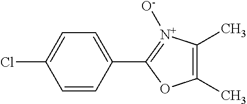

An amount of 1.00 g (9.89 mmol) of diacetylmonoxime and 1.53 g (10.88 mmol) of 4-chlorobenzaldehyde were introduced in 2 ml (34.94 mmol) of glacial acetic acid. Then hydrogen chloride gas was introduced for 30 minutes with accompanying ice cooling of the reaction mixture. Subsequently the reaction mixture was admixed with 10 ml of diethyl ether. A precipitate was produced, which was isolated by filtration with suction and washed with twice 2 ml of diethyl ether. The precipitate was resuspended in about 5 ml of water and the suspension was rendered basic with ammonia. It was then extracted with four times 10 ml of dichloromethane. The combined organic phases were dried over magnesium sulphate and the solvent was removed on a rotary evaporator. The residue was used without further purification in the subsequent reaction.

Yield: 1.85 g (84% of theory)

LC-MS (Method 12): Rₜ=2.29 min; MS (ESIpos): m/z=224 [M+H]⁺.

### Example 26A

### 4-(Chloromethyl)-2-(4-chlorophenyl)-5-methyl-1,3-oxazole

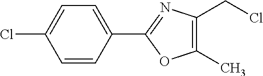

An amount of 1.00 g (4.47 mmol) of the compound from Example 25A was introduced in 15 ml of chloroform and admixed cautiously with 1.5 ml (16.10 mmol) of phosphoryl chloride. The reaction mixture was heated under reflux for 30 minutes with stirring. The batch was subsequently cooled to 0° C. and rendered weakly basic by addition of ammonia. The mixture was extracted with three times 20 ml of ethyl acetate. The combined organic phases were washed with twice 5 ml of water and then dried over magnesium sulphate. The solvent was removed on a rotary evaporator. The residue was used without further purification in the subsequent reaction.

Yield: 1.33 g (96% of theory, 78% purity)

¹H-NMR (400 MHz, DMSO-d₆): δ=7.95 (d, 2H), 7.60 (d, 2H), 4.77 (s, 2H), 2.44 (s, 3H).

### Example 27A

### 2-Amino-6-({[2-(4-chlorophenyl)-5-methyl-1,3-oxazol-4-yl]methyl}sulphanyl)-4-(4-{[4R)-2,2-dimethyl-1,3-dioxolan-4-yl]methoxy}phenyl)pyridine-3,5-dicarbonitrile

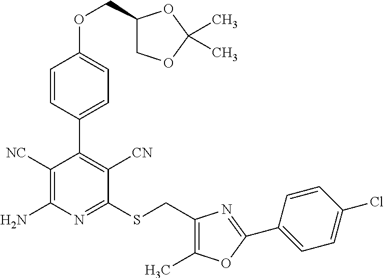

An amount of 4.00 g (10.46 mmol) of the compound from Example 4A and 2.79 g (11.51 mmol) of the compound from Example 26A were suspended together with 2.64 g (31.38 mmol) of sodium hydrogencarbonate in 50 ml of dry DMF. The reaction mixture was stirred at RT overnight. The batch was thereafter admixed with water and stirred further for 30 minutes. The precipitate formed was isolated by filtration with suction and washed with dichloromethane/methanol (3:1). The filtrate was concentrated and the residue was stirred up with dichloromethane/methanol (3:1). The solid that remained was isolated by filtration with suction, combined with the precipitate obtained before, and dried. This gave 5.0 g (81% of theory) of the target compound.

¹H-NMR (400 MHz, DMSO-d₆): δ=8.19-7.97 (br. s, 2H), 7.94 (d, 2H), 7.58 (d, 2H), 7.49 (d, 2H), 7.12 (d, 2H), 4.51 (s, 2H), 4.48-4.41 (m, 1H), 4.16-4.03 (m, 3H), 3.79 (dd, 2H), 2.46 (s, 3H), 1.37 (s, 3H), 1.32 (s, 3H).

LC-MS (Method 7): Rₜ=3.06 min; MS (ESIpos): m/z=588 [M+H]⁺.

### Example 28A

### 2-Amino-6-({[2-(4-chlorophenyl)-5-methyl-1,3-oxazol-4-yl]methyl}sulphanyl)-4-(4-{[(2D)-2,3-dihydroxypropyl]oxy}phenyl)pyridine-3,5-dicarbonitrile

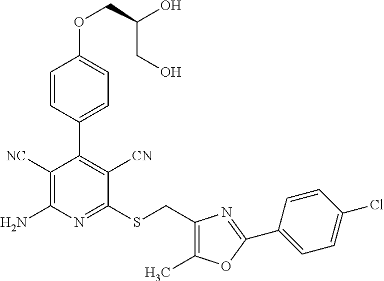

An amount of 5.00 g (8.50 mmol) of the compound from Example 27A was introduced in 800 ml of acetic acid and then admixed cautiously with 100 ml of water. The reaction mixture was stirred at 70° C. for 1 hour. Following removal of the solvent on a rotary evaporator, the residue was dried under a high vacuum. This gave 4.78 g (99% of theory) of the target compound.

¹H-NMR (400 MHz, DMSO-d₆): δ=8.27-7.96 (br. s, 2H), 7.93 (d, 2H), 7.58 (d, 2H), 7.49 (d, 2H), 7.10 (d, 2H), 5.00 (d, 1H), 4.70 (t, 1H), 4.51 (s, 2H), 4.09 (dd, 1H), 3.96 (dd, 1H), 3.84-3.78 (m, 1H), 3.47 (t, 2H), 2.49 (s, 3H).

LC-MS (Method 7): Rₜ=2.50 min; MS (ESIpos): m/z=548 [M+H]⁺.

### Example 29A

### (2R)-3-{4-[2-Amino-6-({[2-(4-chlorophenyl)-5-methyl-1,3-oxazol-4-yl]methyl}sulphanyl)-3,5-dicyanopyridin-4-yl]phenoxy}propane-1,2-diyl(2S,2′S)-bis{2-[(tert-butoxycarbonyl)amino]propanoate}

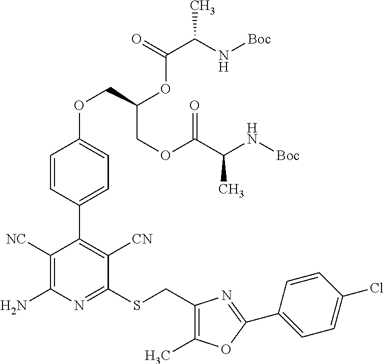

An amount of 2.07 g (10.95 mmol) of N-(tert-butoxycarbonyl)-L-alanine was introduced in 17.5 ml of DMF and admixed in succession with 910 mg (4.74 mmol) of 1-(3-dimethylaminopropyl)-3-ethylcarbodiimide hydrochloride, 223 mg (1.83 mmol) of 4-N,N-dimethylaminopyridine and 1.00 g (1.83 mmol) of the compound from Example 28A. The mixture was stirred at RT for 2 hours and then admixed with water. The mixture was extracted three times with ethyl acetate, and the combined organic phases were dried over magnesium sulphate and concentrated. The crude product was purified by means of preparative HPLC. This gave 771 mg (43% of theory) of the target compound.

LC-MS (Method 5): Rₜ=1.65 min; MS (ESIneg): m/z=888 [M−H]⁻.

### Exemplary Embodiments

### Example 1

### (2S)-3-{4-[2-Amino-6-({[2-(4-chlorophenyl)-1,3-oxazol-4-yl]methyl}sulfanyl)-3,5-dicyanopyridin-4-yl]phenoxy}propane-1,2-diyl(2S,2′S)-bis(2-aminopropanoate)dihydrochloride

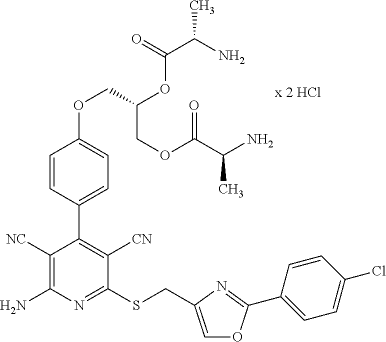

Over 30 minutes, hydrogen chloride gas was introduced into a solution of 6014 mg (6.862 mmol) of the compound from example 10A in 500 ml of dichloromethane, the temperature being held below +20° C. The precipitated solid was isolated by suction filtration, washed with dichloromethane and diethyl ether, and dried under a high vacuum at +80° C. overnight. This gave 5080 mg (99% of theory) of the target compound.

¹H-NMR (400 MHz, DMSO-d₆): δ=8.7 (br. s, 6H), 8.4 (s, 1H), 8.0 (d, 2H), 7.60 (d, 2H), 7.50 (d, 2H), 7.15 (d, 2H), 5.5 (m, 1H), 4.60-4.50 (m, 2H), 4.44 (s, 2H), 4.40 (d, 2H), 4.15 (m, 2H), 1.5-1.4 (m, 6H).

LC-MS (method 7): Rₜ=1.53 min; MS (ESIpos): m/z=676 [M+H]⁺.

### Example 2

### (2S)-3-{4-[2-Amino-6-({[2-(4-chlorophenyl)-1,3-oxazol-4-yl]methyl}sulfanyl)-3,5-dicyanopyridin-4-yl]phenoxy}propane-1,2-diyl (2S,2′S)-bis(2-aminopropanoate)bis(trifluoroacetic acid) salt

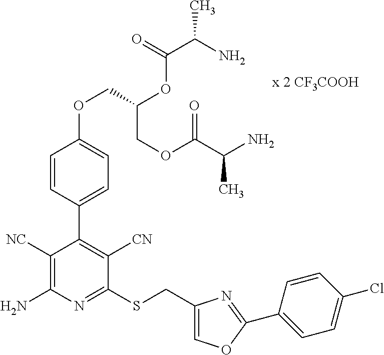

An amount of 6.520 g (7.440 mmol) of the compound from example 10A was introduced in 45 ml of dichloromethane, admixed with 5.732 ml (74.396 mmol) of trifluoroacetic acid and stirred at room temperature overnight. The reaction mixture was then concentrated and the residue was suspended twice with dichloromethane and concentrated again. The residue was then stirred up with diethyl ether, and the solid which remained was isolated by filtration, washed with diethyl ether and dried under a high vacuum. This gave 4.8 g (69% of theory) of the target compound.

¹H-NMR (400 MHz, DMSO-d₆): δ=8.42 (br. s, 4H), 8.36 (s, 1H), 8.19 (m, 2H), 7.97 (d, 2H), 7.60 (d, 2H), 7.50 (d, 2H), 7.13 (d, 2H), 5.52-5.47 (m, 1H), 4.59-4.50 (m, 2H), 4.41 (s, 2H), 4.39-4.31 (m, 2H), 4.19-4.15 (m, 2H), 1.42-1.34 (m, 6H).

LC-MS (method 5): Rₜ=0.94 min; MS (ESIpos): m/z=676 [M+H]⁺.

### Example 3

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis(aminoacetate) dihydrochloride

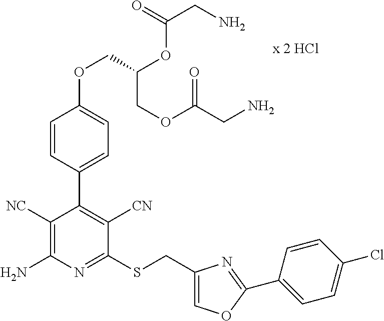

An amount of 440 mg (0.519 mmol) of the compound from Example 11A was admixed with 5 ml of a 4 M solution of hydrogen chloride gas in dioxane and stirred at room temperature for two hours. The precipitated solid was isolated by filtration with suction and dried at +50° C. for four days under reduced pressure. This gave 300 mg (79% of theory) of the target compound.

¹H-NMR (400 MHz, DMSO-d₆): δ=8.47 (br. m, 6H), 8.39 (s, 1H), 8.32-8.02 (br. s, 2H), 7.97 (d, 2H), 7.61 (d, 2H), 7.50 (d, 2H), 7.14 (d, 2H), 5.51 (m, 1H), 4.52 (d, 2H), 4.42 (s, 2H), 4.35 (d, 2H), 3.88 (m, 4H).

LC-MS (Method 5): Rₜ=0.98 min; MS (ESIpos): m/z=648 [M+H]⁺.

### Example 4

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis(5-aminopentanoate)dihydrochloride

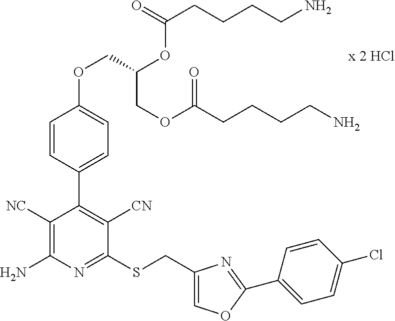

An amount of 200 mg (0.214 mmol) of the compound from Example 12A was introduced in 1.8 ml of dichloromethane and admixed dropwise with 2.2 ml of a 2 M solution of hydrogen chloride gas and diethyl ether. The mixture was stirred at room temperature for an hour and the precipitated solid was then isolated by filtration. It was washed with dichloromethane and dried under reduced pressure. This gave 137 mg (79% of theory) of the target compound.

¹H-NMR (400 MHz, DMSO-d₆): δ=8.39 (s, 1H), 8.35-8.02 (br. s, 2H), 7.97 (d, 2H), 7.93 (br. m, 6H), 7.61 (d, 2H), 7.49 (d, 2H), 7.13 (d, 2H), 5.36 (m, 1H), 4.42 (s, 2H), 4.40 (d, 2H), 4.37 (d, 2H), 2.77 (m, 4H), 2.38 (m, 4H), 1.57 (m, 8H).

LC-MS (Method 6): Rₜ=1.20 min; MS (ESIpos): m/z=732 [M+H]⁺.

### Example 5

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis(2,5-diaminopentanoate)tetrakis(trifluoroacetic acid) salt

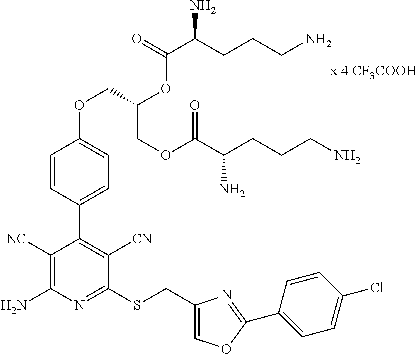

An amount of 276 mg (0.237 mmol) of the compound from Example 13A was introduced in 2 ml of dichloromethane and admixed dropwise with 2.2 ml of a 2 M solution of hydrogen chloride gas in diethyl ether. The mixture was stirred at room temperature for an hour and the precipitated solid was then isolated by filtration. It was washed with dichloromethane and subsequently purified by means of preparative HPLC (eluent: acetonitrile/water+0.1% trifluoroacetic acid). This gave 96 mg (33% of theory) of the target compound.

¹H-NMR (400 MHz, DMSO-d₆): δ=8.56 (br. m, 6H), 8.37 (s, 1H), 8.26-8.06 (br. s, 2H), 7.97 (d, 2H), 7.87 (br. m, 6H), 7.61 (d, 2H), 7.51 (d, 2H), 7.13 (d, 2H), 5.47 (m, 1H), 4.54 (dd, 1H), 4.47-4.30 (m, 5H), 4.16 (br. m, 2H), 2.81 (br. m, 4H), 1.93-1.55 (m, 8H).

LC-MS (Method 7): Rₜ=1.31 min; MS (ESIpos): m/z=762 [M+H]⁺.

### Example 6

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis(2,4-diaminobutanoate)tetrakis(trifluoroacetic acid) salt

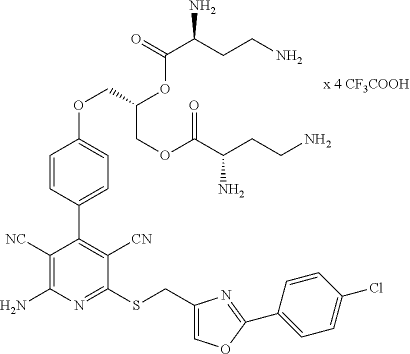

An amount of 255 mg (0.225 mmol) of the compound from Example 14A was introduced in 2 ml of dichloromethane and admixed dropwise with 2.3 ml of a 2 M solution of hydrogen chloride gas in diethyl ether. The mixture was stirred at room temperature for an hour and the precipitated solid was then isolated by filtration. It was washed with dichloromethane and subsequently purified by means of preparative HPLC (eluent: acetonitrile/water+0.1% trifluoroacetic acid). This gave 44 mg (21% of theory) of the target compound.

¹H-NMR (400 MHz, DMSO-d₆): δ=8.66 (br. m, 6H), 8.37 (s, 1H), 8.32-8.12 (br. s, 2H), 8.00 (br. m, 6H), 7.97 (d, 2H), 7.61 (d, 2H), 7.51 (d, 2H), 7.13 (d, 2H), 5.49 (m, 1H), 4.54 (dd, 1H), 4.49 (dd, 1H), 4.42 (s, 2H), 4.40-4.23 (m, 4H), 3.00 (m, 4H), 2.18 (m, 2H), 2.09 (m, 2H).

LC-MS (Method 6): Rₜ=0.88 min; MS (ESIpos): m/z=734 [M+H]⁺.

### Example 7

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis(3-aminopropanoate)dihydrochloride

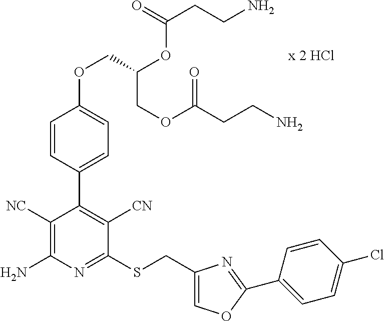

An amount of 123 mg (0.140 mmol) of the compound from example 15A was introduced in 2 ml of dichloromethane and admixed with 1.4 ml (2.807 mmol) of a 2M solution of hydrogen chloride gas in diethyl ether. After 1 hour of stirring, the precipitated solid was isolated by filtration, washed with dichloromethane and diethyl ether and dried under reduced pressure. This gave 79 mg (75% of theory) of the target compound.

¹H-NMR (400 MHz, DMSO-d₆): δ=8.38 (s, 1H), 8.34-8.12 (br. s, 2H), 8.03 (br. m, 6H), 7.97 (d, 2H), 7.61 (d, 2H), 7.49 (d, 2H), 7.14 (d, 2H), 5.40 (m, 1H), 4.42 (s, 2H), 4.41-4.29 (m, 4H), 3.04 (br. m, 4H), 2.75 (br. m, 4H).

LC-MS (Method 6): Rₜ=1.17 min; MS (ESIpos): m/z=676 [M+H]⁺.

### Example 8

### (2R)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis(2,5-diaminopentanoate)-tetrahydro-chloride

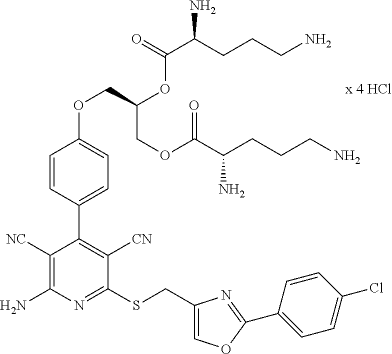

An amount of 233 mg (0.201 mmol) of the compound from Example 16A was introduced in 1.9 ml of dichloromethane and admixed dropwise with 2.01 ml (4.015 mmol) of a 2 M solution of hydrogen chloride gas in diethyl ether. The mixture was stirred at room temperature overnight and then the solvent was removed on a rotary evaporator. The residue was admixed twice in succession with dichloromethane and the solvent was stripped off again each time under reduced pressure. The crude product was dissolved in 2 ml of water and lyophilized. This gave 165 mg (91% of theory) of the target compound.

¹H-NMR (400 MHz, DMSO-d₆): δ=8.82 (br. m, 6H), 8.40 (s, 1H), 8.15 (br. m, 8H), 7.98 (d, 2H), 7.61 (d, 2H), 7.50 (d, 2H), 7.19 (d, 2H), 5.53 (m, 1H), 4.53 (d, 2H), 4.45-4.41 (m, 4H), 4.19 (m, 2H), 2.81 (m, 4H), 2.03-1.64 (m, 8H).

LC-MS (Method 5): Rₜ=0.73 min; MS (ESIpos): m/z=762 [M+H]⁺.

### Example 9

### (2S)-3-{4-[2-Amino-6-({[2-(4-chlorophenyl)-1,3-oxazol-4-yl]methyl}sulfanyl)-3,5-dicyanopyridin-4-yl]phenoxy}propane-1,2-diyl(2R,2′R)-bis(2-aminopropanoate)dihydrochloride

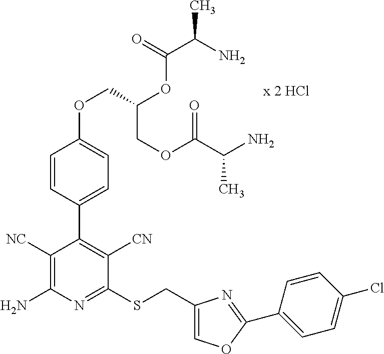

An amount of 675 mg (0.770 mmol) of the compound from example 17A was introduced in 8 ml of dichloromethane and admixed with 15.4 ml of a 1M solution of hydrogen chloride gas in diethyl ether. After 4 hours of stirring, the precipitated solid was isolated by suction filtration, washed with dichloromethane and diethyl ether and dried under reduced pressure. This gave 577 mg (quantitative) of the target compound.

¹H-NMR (400 MHz, DMSO-d₆): δ=8.55 (br. m, 6H), 8.38 (s, 1H), 8.33-8.02 (br. s, 2H), 7.97 (d, 2H), 7.61 (d, 2H), 7.50 (d, 2H), 7.13 (d, 2H), 5.51 (m, 1H), 4.56-4.44 (m, 2H), 4.42 (s, 2H), 4.39-4.33 (m, 2H), 4.16 (m, 2H), 1.44 (d, 3H), 1.39 (d, 3H).

LC-MS (Method 8): Rₜ=0.87 min; MS (ESIpos): m/z=676 [M+H]⁺.

### Example 10

### (2R)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis(2-amino-3-methylbutanoate)-dihydro-chloride

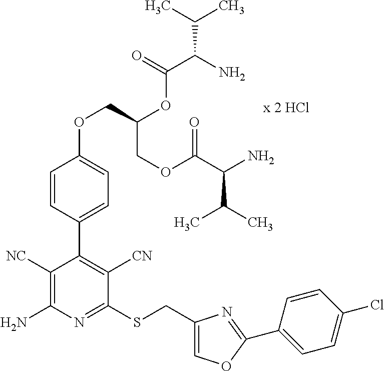

A solution of 48 mg (0.051 mmol) of the compound from Example 18A in 1 ml of dichloromethane was admixed with 20 ml of a saturated solution of hydrogen chloride gas in dichloromethane and stirred at RT for 2 hours. Thereafter the mixture was concentrated and the residue was stirred up with diethyl ether. The solid which remained was isolated by filtration with suction, washed with dichloromethane and with diethyl ether and dried at +80° C. overnight under a high vacuum. This gave 28 mg (65% of theory) of the target compound.

¹H-NMR (400 MHz, DMSO-d₆): δ=8.75-8.6 (br. s, 6H), 8.4 (s, 1H), 7.96 (d, 2H), 7.60 (d, 2H), 7.50 (d, 2H), 7.1 (d, 2H), 5.6 (m, 1H), 4.53 (d, 2H), 4.50-4.35 (m, 4H), 4.1-3.9 (m, 2H), 2.2 (m, 2H), 1.05-0.95 (m, 6H).

LC-MS (Method 7): Rₜ=1.62 min; MS (ESIpos): m/z=732 [M+H]⁺.

### Example 11

### (2R)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis(2-aminopropanoate)dihydrochloride

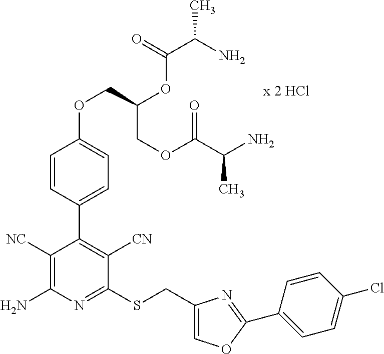

An amount of 1090 mg (1.244 mmol) of the compound from Example 19A was admixed with 125 ml of a saturated solution of hydrogen chloride gas in dichloromethane and treated in an ultrasound bath for 2 hours. Thereafter the mixture was concentrated and the residue was stirred up with diethyl ether. The solid which remained was isolated by filtration with suction, washed twice with diethyl ether and dried at +80° C. overnight under a high vacuum. This gave 770 mg (79% of theory) of the target compound.

¹H-NMR (400 MHz, DMSO-d₆): δ=8.7-8.5 (br. s, 6H), 8.35 (s, 1H), 8.45 (d, 2H), 7.6 (d, 2H), 7.5 (d, 2H), 7.13 (d, 2H), 5.5 (m, 1H), 4.59-4.30 (m, 6H), 1.45 (d, 3H), 1.40 (d, 3H).

LC-MS (Method 5): Rₜ=0.99 min; MS (ESIpos): m/z=676 [M+H]⁺.

### Example 12

### (2R)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis(4-aminobutanoate)dihydrochloride

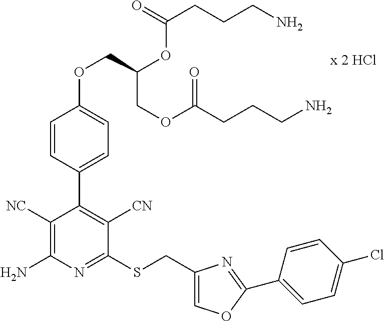

An amount of 70 mg (0.077 mmol) of the compound from Example 20A was admixed with 10 ml of a saturated solution of hydrogen chloride gas in dichloromethane and treated in an ultrasound bath for 2 hours. Thereafter the mixture was concentrated and the residue was stirred up with diethyl ether. The solid which remained was isolated by filtration with suction, washed twice with diethyl ether and dried at +80° C. overnight under a high vacuum. This gave 47 mg (75% of theory) of the target compound.

¹H-NMR (400 MHz, DMSO-d₆): δ=8.4 (s, 1H), 8.1-8.0 (br. s, 6H), 7.95 (d, 2H), 7.6 (d, 2H), 7.45 (d, 2H), 7.1 (d, 2H), 5.4 (m, 1H), 4.45-4.25 (m, 6H), 2.9-2.8 (m, 4H), 2.5-2.4 (m, 4H), 1.9-1.8 (m, 4H).

LC-MS (Method 7): Rₜ=1.47 min; MS (ESIpos): m/z=704 [M+H]⁺.

### Example 13

### (2S)-3-{4-[2-Amino-6-({[2-(4-chlorophenyl)-1,3-oxazol-4-yl]methyl}sulphanyl)-3,5-dicyanopyridin-4-yl]phenoxy)propane-1,2-diyl(2S,2′S)-bis(2,6-diaminohexanoate)dihydrochloride

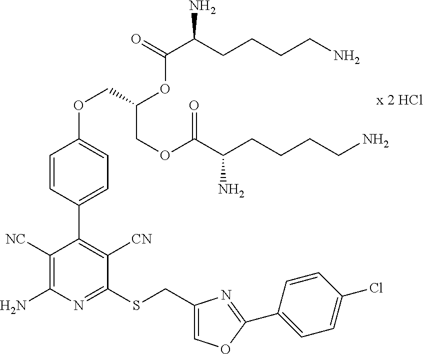

Hydrogen chloride gas was introduced for 1 hour into a solution of 425 mg (0.357 mmol) of the compound from Example 21A in 200 ml of dichloromethane, the temperature being held below +20° C. Subsequently the mixture was stirred at RT for a further 2 hours. After that it was concentrated under reduced pressure and the residue which remained was taken up in 200 ml of water. It was extracted by shaking twice each with dichloromethane and ethyl acetate. The aqueous phase was filtered and then concentrated under reduced pressure to half its volume. Lyophilisation of the solution gave 277 mg (83% of theory) of the target compound.

¹H-NMR (400 MHz, DMSO-d₆): δ=8.9-8.7 (m, 6H), 8.38 (s, 1H), 8.2-8.0 (m, 6H), 7.9 (d, 2H), 7.6 (d, 2H), 7.48 (d, 2H), 7.15 (d, 2H), 5.5 (m, 1H), 4.6-4.4 (m, 6H), 4.1-4.0 (m, 2H), 2.8-2.7 (m, 4H), 1.9-1.8 (m, 4H), 1.7-1.4 (m, 8H).

LC-MS (Method 7): Rₜ=1.01 min; MS (ESIpos): m/z=790 [M+H]⁺.

### Example 14

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis(4-aminobutanoate)dihydrochloride

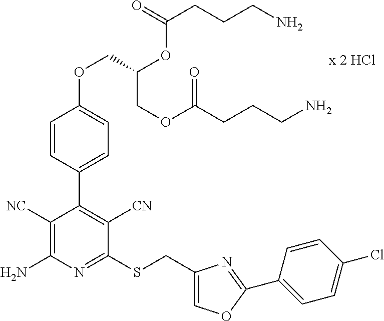

Hydrogen chloride gas was passed for 1 hour into a solution of 290 mg (0.321 mmol) of the compound from Example 22A in 500 ml of dichloromethane, the temperature being held below +20° C. Subsequently the mixture was stirred at RT for a further 6 hours. After that it was concentrated under reduced pressure. The residue was taken up in 10 ml of acetonitrile/water (1:5) and purified by means of preparative HPLC. The product fractions were combined and concentrated. The residue was taken up in hydrochloric acid, which was set at a pH of 3, and the solution was subsequently lyophilised. This gave 90 mg (36% of theory) of the target compound.

¹H-NMR (400 MHz, DMSO-d₆): δ=8.38 (s, 1H), 8.0 (br. s, 6H), 7.95 (d, 2H), 7.6 (d, 2H), 7.45 (d, 2H), 7.1 (d,.2H), 5.38 (m, 1H), 4.45-4.25 (m, 6H), 2.85-2.75 (m, 4H), 1.9-1.8 (m, 4H).

LC-MS (Method 7): Rₜ=1.51 min; MS (ESIpos): m/z=704 [M+H]⁺.

### Example 15

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl bis(6-aminohexanoate) dihydrochloride

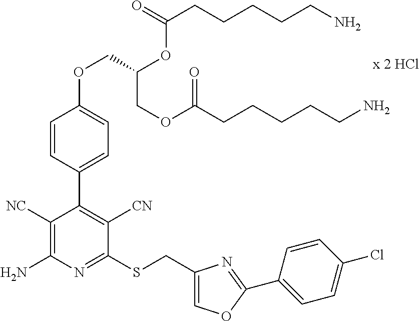

Hydrogen chloride gas was passed for 1 hour into a solution of 275 mg (0.286 mmol) of the compound from Example 23A in 500 ml dichloromethane, the temperature being held below +20° C. Subsequently the mixture was stirred at RT for a further 6 hours. After that it was concentrated under reduced pressure. The residue was taken up in 10 ml of acetonitrile/water (1:5) and purified by means of preparative HPLC. The product fractions were combined and concentrated. The residue was taken up in hydrochloric acid, which was set at a pH of 3, and the solution was subsequently lyophilised. This gave 187 mg (78% of theory) of the target compound.

¹H-NMR (400 MHz, DMSO-d₆): δ=8.4 (s, 1H), 7.95 (d, 2H), 7.9-7.7 (br. s, 6H), 7.6 (d, 2H), 7.45 (d, 2H), 7.1 (d, 2H), 5.35 (m, 1H), 4.45-4.2 (m, 6H), 2.8-2.7 (m, 4H), 2.32 (t, 4H), 1.6-1.5 (m, 8H), 1.35-1.25 (m, 4H).

LC-MS (Method 7): Rₜ=1.58 min; MS (ESIpos): m/z=760 [M+H]⁺.

### Example 16

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis(2-amino-3-methylbutanoate)bis(trifluoroacetic acid) salt

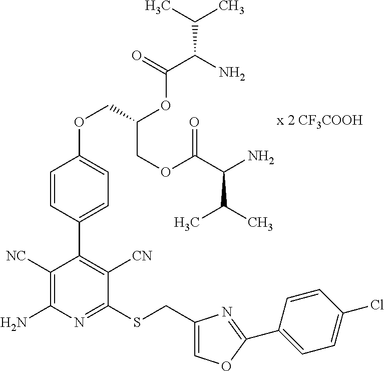

An amount of 400 mg (0.75 mmol) of the compound from Example 8A, 651 mg (3 mmol) of N-Boc-L-valine, 718 mg (3.745 mmol) of 1-(3-dimethylaminopropyl)-3-ethylcarbodiimide hydrochloride and 91.5 mg (0.75 mmol) of 4-N,N-dimethylaminopyridine were combined under argon in 40 ml of dichloromethane and treated in an ultrasound bath for 30 minutes. The batch was then extracted by shaking with four times 20 ml of 50% strength sodium hydrogencarbonate solution and subsequently with four times 20 ml of 0.5 M citric acid solution. The organic phase was dried over magnesium sulphate and concentrated. The residue was stirred up with cyclohexane and the solid which remained was isolated by filtration with suction. It was dissolved in dichloromethane and precipitated again with cyclohexane. The precipitate this time was isolated by filtration with suction and dried under reduced pressure. This gave 801 mg of the boc-protected intermediate.

This intermediate was taken up in 70 ml of dichloromethane, admixed dropwise with 7 ml of anhydrous trifluoroacetic acid and stirred at RT for 30 minutes. Thereafter the mixture was concentrated under a high vacuum. The residue was stirred up with methyl tert-butyl ether, and the solid was isolated by filtration with suction and subsequently recrystallized from hot isopropanol. After further isolation by filtration with suction and after drying of the filter residue under a high vacuum, 360 mg (47% of theory) of the title compound were obtained.

LC-MS (Method 6): Rₜ=1.24 min; MS (ESIpos): m/z=732 [M+H]⁺.

### Example 17

### (2S)-3-{4-[2-Amino-6-({[2-(4-chlorophenyl)-1,3-oxazol-4-yl]methyl}sulphanyl)-3,5-dicyanopyridin-4-yl]phenoxy}propane-1,2-diyl(2S,2′S)-bis(2-amino-4-methylpentanoate)bis(trifluoroacetic acid) salt

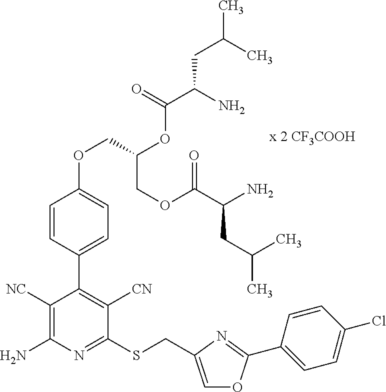

An amount of 400 mg (0.75 mmol) of the compound from Example 8A, 693 mg (3 mmol) of N-Boc-L-leucine, 718 mg (3.745 mmol) of 1-(3-dimethylaminopropyl)-3-ethylcarbodiimide hydrochloride and 91.5 mg (0.75 mmol) of 4-N,N-dimethylaminopyridine were combined under argon in 40 ml of dichloromethane and treated in an ultrasound bath for 30 minutes. The batch was then extracted by shaking with five times 20 ml of 0.5 M citric acid solution and subsequently with five times 20 ml of 50% strength sodium hydrogen carbonate solution. The organic phase was dried over magnesium sulphate and concentrated. The residue was taken up in dichloromethane and precipitated with cyclohexane. The precipitate was isolated by filtration with suction and dried under reduced pressure. This gave 715 mg of the boc-protected intermediate. This intermediate was taken up in 70 ml of dichloromethane, admixed dropwise with 7 ml of anhydrous trifluoroacetic acid and stirred at RT for 75 minutes. Thereafter the mixture was concentrated under a high vacuum. The residue was stirred up for 2 days in 50 ml of methyl tert-butyl ether, and the solid was then isolated by filtration with suction and subsequently recrystallized from hot isopropanol. After further isolation by filtration with suction and after the drying of the filter residue under a high vacuum, 512 mg (69% of theory) of the title compound were obtained.

LC-MS (Method 5): Rₜ=1.08 min; MS (ESIpos): m/z=760 [M+H]⁺.

### Example 18

### (2S)-3-{4-[2-Amino-6-({[2-(4-chlorophenyl)-1,3-oxazol-4-yl]methyl}sulfanyl)-3,5-dicyanopyridin-4-yl]phenoxy}propane-1,2-diyl(2S,3R,2′S,3′R)-bis(2-amino-3-hydroxybutanoate)bis(trifluoroacetic acid) salt

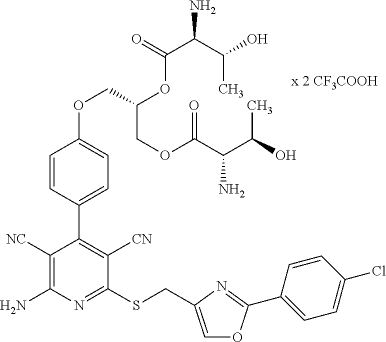

An amount of 916 mg (0.873 mmol) of the compound from Example 24A was introduced in 10 ml of dichloromethane, admixed with 1.35 ml (17.47 mmol) of trifluoroacetic acid and stirred at room temperature overnight. The reaction mixture was subsequently concentrated and the residue was purified by means of preparative HPLC (eluent: acetonitrile/water+0.1% trifluoroacetic acid). This gave 574 mg (68% of theory) of the target compound.

¹H-NMR (400 MHz, DMSO-d₆): δ=8.42-8.25 (m, 5H), 8.15 (br. s, 2H), 7.98 (d, 2H), 7.61 (d, 2H), 7.51 (d, 2H), 7.12 (d, 2H), 5.68 (br. s, 1H), 5.50 (quint, 1H), 4.50 (d, 2H), 4.42 (s, 2H), 4.38-4.32 (m, 2H), 4.24-3.95 (m, 5H), 1.24-1.18 (m, 6H).

LC-MS (Method 7): Rₜ=1.45 min; MS (ESIpos): m/z=736 [M+H]⁺.

### Example 19

### (2R)-3-{4-[2-Amino-6-({[2-(4-chlorophenyl)-5-methyl-1,3-oxazol-4-yl]methyl}sulphanyl)-3,5-dicyanopyridin-4-yl]phenoxy}propane-1,2-diyl-(2S,2′S)-bis(2-aminopropanoate)-dihydrochloride

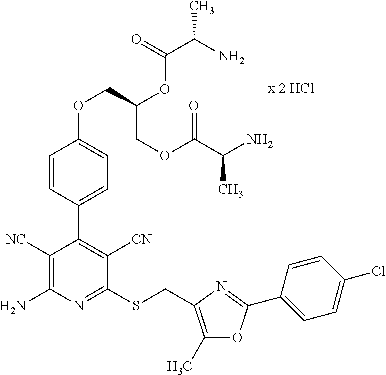

An amount of 3.1 g (3.48 mmol) of the compound from Example 29A was introduced in 32 ml of dichloromethane and admixed dropwise with 17 ml of a 2 M solution of hydrogen chloride gas in diethyl ether. The mixture was stirred at room temperature for six hours. The precipitated solid was isolated by filtration, washed with dichloromethane and dried under reduced pressure. This gave 2.65 g (98% of theory) of the title compound.

¹H-NMR (400 MHz, DMSO-d₆): δ=8.80-8.50 (m, 6H), 8.10 (br. s, 2H), 7.92 (d, 2H), 7.59 (d, 2H), 7.51 (d, 2H), 7.14 (d, 2H), 5.56-5.48 (m, 1H), 4.58-4.32 (m, 6H), 4.22-4.09 (m, 2H), 2.55 (s, 3H), 1.45 (d, 3H), 1.41 (d, 3H).

LC-MS (Method 7): Rₜ=1.73 min; MS (ESIpos): m/z=690 [M+H]⁺.

### B. DETERMINATION OF SOLUBILITY, STABILITY AND LIBERATION BEHAVIOR

a) Determination of the Solubility:

The test substance is suspended in water or dilute hydrochloric acid (pH 4) or in 5% strength aqueous dextrose solution. This suspension is shaken at room temperature for 24 h. After ultracentrifugation at 224 000 g for 30 min, the supernatant is diluted with DMSO and analysed by HPLC. A two-point calibration plot of the test compound in DMSO is used for quantification.

HPLC method for acids:

Agilent 1100 with DAD (G1315A), quat. Pump (G1311A), autosampler CTC HTS PAL, degasser (G1322A) and column thermostat (G1316A); column: Phenomenex Gemini C18, 5 μm, 50 mm×2 mm; temperature: 40° C.; eluent A: water/phosphoric acid pH 2, eluent B: acetonitrile; flow rate: 0.7 ml/min; gradient: 0-0.5 min 85% A, 15% B; ramp 0.5-3 min 10% A, 90% B; 3-3.5 min 10% A, 90% B; ramp 3.5-4 min 85% A, 15% B; 4-5 min 85% A, 15% B.

HPLC method for bases:

Agilent 1100 with DAD (G1315A), quat. pump (G1311A), autosampler CTC HTS PAL, degasser (G1322A) and column thermostat (G1316A); column: VDSoptilab Kromasil 100 C18, 3.5 μm, 60 mm×2.1 mm; temperature: 30° C.; eluent A: water+5 ml of perchloric acid/liter, eluent B: acetonitrile; flow rate: 0.75 ml/min; gradient: 0-0.5 min 98% A, 2% B; ramp 0.5-4.5 min 10% A, 90% B; 4.5-6 min 10% A, 90% B; ramp 6.5-6.7 min 98% A, 2% B; 6.7-7.5 min 98% A, 2% B.

The solubilities of representative exemplary embodiments in dilute hydrochloric acid (pH 4) are shown in Table1:

TABLE 1

| Example No. | Solubility [mg/liter] |
|---|---|
| 1 | >500 |
| 9 | 535 |

The solubilities of representative exemplary embodiments in 5% strength aqueous dextrose solution are shown in Table 2:

TABLE 2

| Example No. | Solubility [mg/liter] |
|---|---|
| 1 | >500 |
| 10 | >500 |
| 13 | 710 |
| 14 | 540 |
| 15 | 830 |
| 16 | 250 |
| 17 | 330 |
| 18 | 240 |

No decomposition of the exemplary compounds in these solutions was observed. The solubility of the active substance from example 8A was determined in 0.1 M hydrochloric acid to be <0.1 mg/liter and in 5% strength aqueous dextrose solution to be <1.2 mg/liter.

b) Stability in Buffer at Various pH Values:

0.3 mg of the test substance is weighed into a 2 ml HPLC vial and 0.5 ml of acetonitrile or acetonitrile/DMSO (9:1) is added. The substance is dissolved by putting the sample vessel in an ultrasonic bath for about 10 seconds. Then 0.5 ml of the respective buffer solution is added, and the sample is again treated in the ultrasonic bath.

(Buffer) solutions employed:

pH 4: 1 liter of Millipore water is adjusted to pH 4.0 with 1 N hydrochloric acid;

pH 5: 0.096 mol of citric acid and 0.2 mol of sodium hydroxide ad 1 liter of water;

pH 7.4: 90.0 g of sodium chloride, 13.61 g of potassium dihydrogen phosphate and 83.35 g of 1 N sodium hydroxide solution are made up to 1 liter with water; this solution is then further diluted 1:10 with Millipore water.

pH 8: 0.013 mol of borax and 0.021 mol of hydrochloric acid ad 1 liter of water.

5 μl portions of the test solution are analyzed by HPLC for their content of unchanged test substance, and of basic mother substance of the formula (A) formed, every hour over a period of 24 hours at 37° C. The percentage areas of the appropriate peaks are used for quantification.

HPLC method for example 1:

Agilent 1100 with DAD (G1315B), binary pump (G1312A), autosampler (G1329A), column oven (G1316A), thermostat (G1330B); column: Kromasil 100 C18, 125 mm×4 mm, 5 μm; column temperature: 30° C.; eluent A: water+5 ml perchloric acid/liter, eluent B: acetonitrile; gradient: 0 min 90% A→2.0 min 70% A→18.0 min 70% A 20.0 min 10% A→21.0 min 10% A→23.0 min 90% A→26.0 min 90% A; flow rate: 2.0 ml/min;

UV detetection: 294 nm.

HPLC method for example 3:

Agilent 1100 with DAD (G1314A), binary pump (G1312A), autosampler (G1329A), column oven (G1316A), thermostat (G1330A); column: Kromasil 100 C18, 125 mm×4 mm, 5 μm; column temperature: 30° C.; eluent A: water+5 ml perchloric acid/liter, eluent B: acetonitrile; gradient: 0 min 90% A→2.0 min 64% A→18.0 min 64% A→20.0 min 10% A→21.0 min 10% A→23.0 min 90% A→26.0 min 90% A; flow rate: 2.0 ml/min UV detection: 294 nm.

HPLC method for example 11:

Agilent 1100 with DAD (G1315B), binary pump (G1312A), autosampler (G1329A), column oven (G1316A), thermostat (G1330B); column: Kromasil 100 C18, 125 mm×4 mm, 5 μm; column temperature: 30° C.; eluent A: water+5 ml of perchloric acid/liter, eluent B: acetonitrile; gradient: 0 min 90% A→2.0 min 64% A→18.0 min 64% A→20.0 min 10% A→21.0 min 10% A→23.0 min 90% A→26.0 min 90% A; flow rate: 2.0 ml/min; UV detection: 294 nm.

HPLC method for example 14:

Agilent 1100 with DAD (G1315B), binary pump (G1312A), autosampler (G1329A), column oven (G1316A), thermostat (G1330B); column: Kromasil 100 C18, 250 mm×4 mm, 5 μm; column temperature: 30° C.; eluent A: water+5 ml of perchloric acid/liter, eluent B: acetonitrile; gradient: 0 min 90% A→7.0 min 52% A→18.0 min 52% A→20.0 min 10% A→21.0 min 10% A→23.0 min 90% A -4 26.0 min 90% A; flow rate: 2.0 ml/min; UV detection: 288 nm.

HPLC method for example 18:

Agilent 1100 with DAD (G1315A), binary pump (G1312A), autosampler (G1329A), column oven (G1316A), thermostat (G1330A); column: Kromasil 100 C18, 125 mm×4 mm, 5 μm; column temperature: 30° C.; eluent A: water+5 ml of perchloric acid/liter, eluent B: acetonitrile; gradient: 0 min 90% A→6.0 min 61% A→18.0 min 61% A→20.0 min 10% A→21.0 min 10% A→23.0 min 90% A→26.0 min 90% A; flow rate: 2.0 ml/min; UV detection: 294 nm.

The ratios of the peak areas (F) at the respective time points in relation to the peak areas at the starting time are shown in Table 3 for representative exemplary embodiments:

TABLE 3

| | | % test substance | % test substance |
|---|---|---|---|
| | | after 4 h | after 24 h |
| Example | | [F(t = 4 h) × 100/ | [F(t = 24 h) × 100/ |
| No. | pH | F(t = 0 h)] | F(t = 0 h)] |
| 1 | 4 | 99 | 98 |
| 1 | 7.4 | 90 | 53 |
| 3 | 4 | 99 | 99 |
| 3 | 7.4 | 93 | 67 |
| 11 | 4 | 96 | 96 |
| 11 | 7.4 | 84 | 44 |
| 14 | 4 | 97 | 92 |
| 14 | 7.4 | 93 | 74 |
| 18 | 4 | 100 | 95 |
| 18 | 7.4 | 95 | 72 |

In this test there was found to be a decrease in the content of test substance at the same time as an increase in the active ingredient compound from example 8A and 9A in question.

c) In vitro Stability in Rat and Human Plasma:

1 mg of the test substance is weighed into a 2 ml HPLC vial, and 1.5 ml of DMSO and 1 ml of water are added. The substance is dissolved by placing the sample vessel in an ultrasonic bath for about 10 seconds. 0.5 ml of rat or human plasma at 37° C. is added to 0.5 ml of this solution. The sample is shaken, and about 10 μl are removed for a first analysis (time point tₒ). 4-6 further aliquots are removed for quantification in the period up to 2 hours after the start of incubation. The sample is kept at 37° C. during the time of the test. Characterization and quantification take place by HPLC.

HPLC method:

Agilent 1100 with DAD (G1314A), binary pump (G1312A), autosampler (G1329A), column oven (G1316A), thermostat (G1330A); column: Kromasil 100 C18, 250 mm×4 mm, 5 μm; column temperature: 30° C.; eluent A: water+5 ml of perchloric acid/liter, eluent B: acetonitrile; gradient: 0-8.0 min 53% A, 47% B; 8.0-18.0 min 53% A, 47% B; 18.0-20.0 min 90% A, 10% B; 20.0-21.0 min 90% A, 10% B; 21.0-22.5 min 98% A, 2%, B; 22.5-25.0 min 98% A, 2% B; flow rate: 2 ml/min; UV detection: 294 nm. Table 4 indicates the respective times for representative exemplary embodiments at which 50% of the maximum possible amount of active ingredient compound (example 8A and 9A) have been produced (t_{50%A}) after incubation with rat plasma. For the evaluation, the ratio of the peak areas at the individual time points compared with the starting time point was used in each case.

TABLE 4

| | t_{50% A} [min] |
|---|---|
| Example No. | in rat plasma |
| 1 | 5 |
| 3 | 10 |
| 11 | 5 |
| 13 | 30 |
| 14 | 60 |
| 15 | 30 |

d) i.v. Pharmacokinetics in Wistar Rats:

On the day before administration of the substance, a catheter for obtaining blood is implanted in the jugular vein of the experimental animals (male Wistar rats, body weight 200-250 g) under Isofluran® anesthesia.

On the day of the experiment, a defined dose of the test substance is administered as solution into the tail vein using a Hamilton® glass syringe (bolus administration, duration of administration <10 s). Blood samples (8-12 time points) are taken through the catheter sequentially over the course of 24 h after administration of the substance.

Plasma is obtained by centrifuging the samples in heparinized tubes. Acetonitrile is added to a defined plasma volume per time point to precipitate proteins. After centrifugation, test substance and, where appropriate, known cleavage products of the test substance in the supernatant are determined quantitatively using a suitable LC/MS-MS method.

The measured plasma concentrations are used to calculate pharmacokinetic parameters of the test substance and of the active ingredient compound (A) liberated therefrom, such as AUC, Cₘₐₓ, T_{1/2} (half-life) and CL (clearance).** After i.v. administration of the compound from example 1, the substance was no longer detectable in plasma even at the first measurement point. Only the active ingredient (example 8A) was detectable up to the 8-hour time point too.

e) Hemodynamic Measurements on Anesthetized Rats:

Wistar rats (250-300 g body weight; from Harlan-Winkelmann) are anesthetized with 5% Isofluran®. Anesthesia is maintained with 2% Isofluran® and pressurized air in an anesthesia mask. The carotid artery is exposed, and a tip catheter (Millar micro-tip transducer, 2 French; from HSE) is inserted and advanced into the left ventricle. A second catheter is then inserted into the jugular vein. Through this catheter, placebo solution and test substance solutions in increasing concentration are infused into the animals. At the same time, the cardiac function (such as heart rate, left ventricular pressure, contractility (dp/dt), left ventricular end-diastolic pressure) is measured via the left ventricular catheter. By withdrawing the catheter from the left ventricle into the aorta, it is also possible to measure the systemic blood pressure as well.

Exemplary embodiments of pharmaceutical compositions

The compounds of the invention can be converted into pharmaceutical preparations in the following ways:

Tablet:

Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

Production:

The mixture of the compound of the invention, lactose and starch is granulated with a 5% strength solution (m/m) of PVP in water. The granules are dried and then mixed with the magnesium stearate for 5 min. This mixture is compressed with a conventional tablet press (see above for format of the tablet). As guideline, a compressive force of 15 kN is used for the compression.

Oral suspension:

Composition:

1000 mg of the compound of the invention, 1000 mg of ethanol (96%), 400 mg of Rhodigel® (xanthan gum from FMC, Pennsylvania, USA) and 99 g of water.

10 ml of oral suspension are equivalent to a single dose of 100 mg of the compound of the invention.

Production:

The Rhodigel is suspended in ethanol, and the compound of the invention is added to the suspension. The water is added while stirring. The mixture is stirred for about 6 hours until the swelling of the Rhodigel is complete.

Oral solution:

Composition:

500 mg of the compound of the invention, 2.5 g of polysorbate and 97 g of polyethylene glycol 400. A single dose of 100 mg of the compound of the invention corresponds to 20 g oral solution.

Production:

The compound of the invention is suspended in a mixture of polyethylene glycol and polysorbate with stirring. The stirring is continued until the compound of the invention has completely dissolved.

i.v. solution:

The compound of the invention is dissolved in a concentration below the saturation solubility in a physiologically tolerated solvent (e.g. isotronic saline solution, 5% glucose solution and/or 30% PEG 400 solution, in each case adjusted to a pH of 3-5). The solution is optionally filtered sterile and/or dispensed into sterile and pyrogen-free injection containers.

1. A compound of the formula (I) 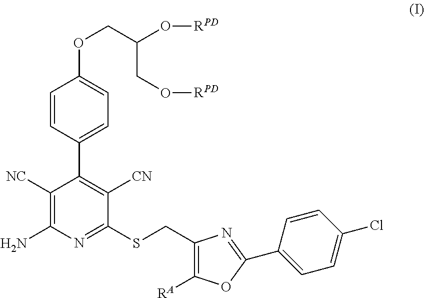
in which
R^{A} is hydrogen or methyl
and
R^{PD} is a group of the formula 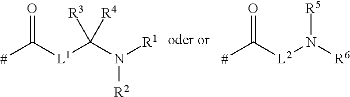
in which
# means the point of linkage to the respective O atom,
L¹ is a bond or —CH₂—,
L² is straight-chain (C₃-C₆)-alkanediyl, which may be substituted up to two times, identically or differently by (C₁-C₄)-alkyl, hydroxyl and/or (C₁-C₄)-alkoxy,
R¹ and R² are identical or different and independently of one another are hydrogen or (C₁-C₄)-alkyl which may be substituted by hydroxyl, (C₁-C₄)-alkoxy, amino, mono-(C₁-C₄)-alkylamino or di-(C₁-C₄)-alkylamino
or
R¹ and R² are linked to one another and, together with the nitrogen atom to which they are attached, form a 5- or 6-membered saturated heterocycle which may comprise a further ring heteroatom from the series consisting of N and O, and may be substituted one or two times, identically or differently, by (C₁-C₄)-alkyl, amino, hydroxyl and/or (C₁-C₄)-alkoxy,
R³ is hydrogen or the side group of a natural α-amino acid or its homologs or isomers,
or
R³ is linked to R¹ and both, together with the atoms to which they are attached, form a 5- or 6-membered saturated heterocycle which may be substituted once or twice by identical or different (C₁-C₄)-alkyl, amino, hydroxyl and/or (C₁-C₄)-alkoxy substituents,
R⁴ is hydrogen or methyl
or
R³ and R⁴ are linked to one another and, together with the carbon atom to which they are attached, form a 3- to 6-membered saturated carbocycle,
and
R⁵ and R⁶ are identical or different and independently of one another are hydrogen or (C₁-C₄)-alkyl which may be substituted by hydroxyl, (C₁-C₄)-alkoxy, amino, mono-(C₁-C₄)-alkylamino or di-(C₁-C₄)-alkylamino,
and the salts thereof.

2. The compound of the formula (I) as claimed in claim 1, in which
R^{A} is hydrogen or methyl
and
R^{PD} is a group of the formula 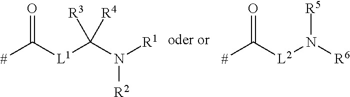
in which
# means the point of linkage to the respective O atom,
L¹ is a bond or —CH₂—,
L² is straight-chain (C₃-C₆)-alkanediyl,
R¹ and R² are independently of one another hydrogen or methyl,
R³ is hydrogen, methyl, propan-2-yl, 2-methylpropan-1-yl, benzyl, imidazol-4-ylmethyl, hydroxymethyl, 1-hydroxyethyl, 4-aminobutan-1-yl, 3-aminopropan-1-yl, 2-aminoethyl, aminomethyl or 3-guanidinopropan-1-yl
or
R³ is linked to R¹ and both, together with the atoms to which they are attached, form a pyrrolidine ring,
R⁴ is hydrogen
and
R⁵ and R⁶ are independently of one another hydrogen or methyl,
and the salts thereof.

3. The compound of the formula (I) as claimed in claim 1, in which
R^{A} is hydrogen
and
R^{PD} is a group of the formula 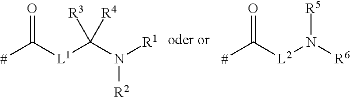
in which
# means the point of linkage to the respective O atom,
L¹ is a bond,
L² is straight-chain (C₃-C₅)-alkanediyl,
R¹ and R² are in each case hydrogen,
R³ is hydrogen, methyl, propan-2-yl, 2-methylpropan-1-yl, hydroxymethyl, 1-hydroxyethyl, 4-aminobutan-1-yl, 3-aminopropan-1-yl, 2-aminoethyl, aminomethyl or 3-guanidinopropan-1-yl,
R⁴ is hydrogen
and
R⁵ and R⁶ are in each case hydrogen,
and the salts thereof.

4. The compound of the formula (I) as claimed in claim 1, in which the two groups R^{PD} are identical, and the salts thereof.

5. The compound as claimed in claim 1, with the formula (I-A) 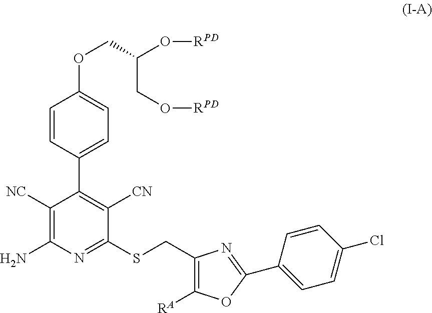
and the salts thereof.

6. A process for preparing compounds of the formula (I) as defined in claim 1, in which the two groups R^{PD} are each identical, characterized in that
[A] the compound of the formula (A) 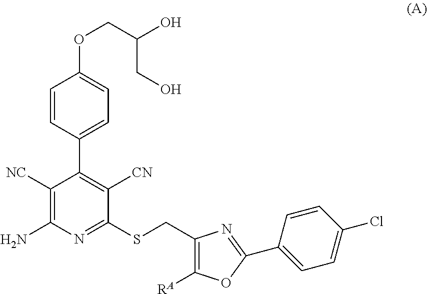
in which
R^{A} is hydrogen or methyl
is coupled in an inert solvent with two or more equivalents of a compound of the formula (II) 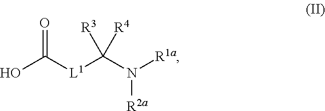
in which L¹, R³ and R⁴ have the meanings indicated in claim 1
and
R^{1a} and R^{2a} are identical or different and have the meanings indicated in claim 1 for
R¹ and R², respectively, or are a temporary amino-protective group, with activation of the carboxyl group in (II), to give a compound of the formula (III) 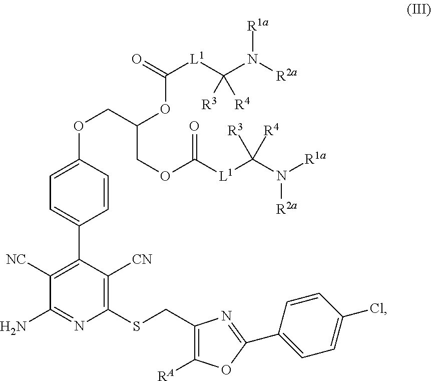
in which L¹, R^{A}, R^{1a}, R^{2a}, R³ and R⁴ have the meanings indicated above, and then any protective groups present are removed, to give a compound of the formula (I-C) 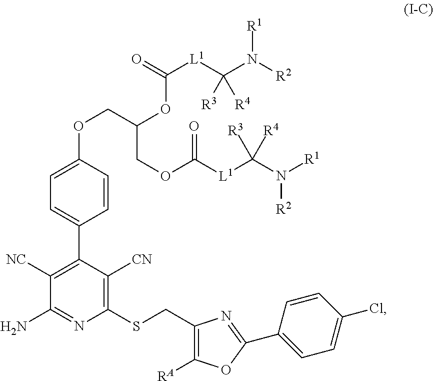
in which L¹, R^{A}, R¹, R², R³ and R⁴ have the meanings indicated in claim 1,
or
[B] the compound of the formula (A) is coupled in an inert solvent with two or more equivalents of a compound of the formula (IV) 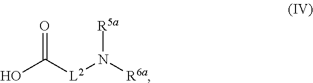
in which L² has the meaning indicated in claim 1
and
R^{5a} and R^{6a} are identical or different and have the meanings indicated in claim 1 for
R⁵ and R⁶, respectively, or are a temporary amino-protective group, with activation of the carboxyl group in (IV), to give a compound of the formula (V) 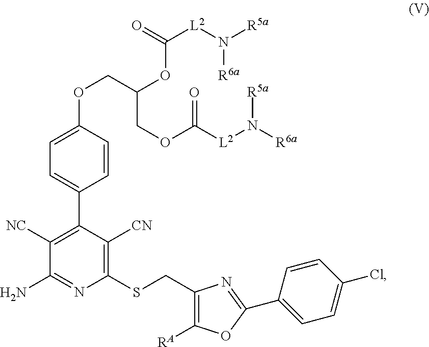
and then any protective groups present are removed, to give a compound of the formula (I-D) 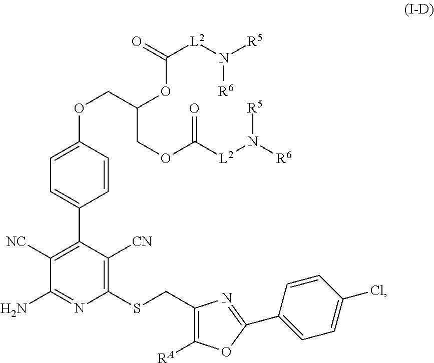
in which L², R⁴, R^{5a} and R^{6a} have the meanings indicated in claim 1,
and the resulting compounds of the formula (I-C) or (I-D) are optionally converted with the appropriate (i) solvents and/or (ii) acids or bases into the salts thereof.

7-9. (canceled)

10. A medicament comprising a compound as defined in claim 1 and an inert, non-toxic, pharmaceutically suitable excipient.

11-13. (canceled)

14. A method of treating or reducing the risk of angina pectoris, atrial fibrillation, hypertension, acute coronary syndrome, coronary heart disease, heart failure, ischemic damage to the heart, post myocardial infarction angina pectoris, secondary myocardial infarction, or worsening heart failure, comprising the step of administering a compound as define in claim 1 to a patient.
